(19) United States
(12) Patent Application Publication
(10) Pub. No.: US 2012/0277257 A1
(43) Pub. Date: Nov. 1, 2012

(54) Methods of Treating Diseases, Pharmaceutical Compositions, and Pharmaceutical Dosage Forms

(75) Inventors: Margaret YU, Sherman Oaks, CA (US); Daniel A. WETTSTEIN, Salt Lake City, UT (US); Vijay R. BAICHWAL, Foster City, CA (US); Damon I. PAPAC, Mountain Brook, AL (US); Gaylen M. ZENTNER, Salt Lake City, UT (US); Mark S. WILLIAMS, Lee's Summit, MO (US)

(73) Assignee: MYREXIS, INC., Salt Lake City, UT (US)

(21) Appl. No.: 13/470,914

(22) Filed: May 14, 2012

Related U.S. Application Data
(63) Continuation of application No. PCT/US2010/056522, filed on Nov. 12, 2010.
(60) Provisional application No. 61/261,258, filed on Nov. 13, 2009.
(60) Provisional application No. 61/285,882, filed on Dec. 11, 2009.
(60) Provisional application No. 61/324,666, filed on Apr. 15, 2010.

(51) Int. Cl. A61K 31/52; A61P 35/00; B29B 9/00; A61P 35/02

(52) U.S. Cl. 514/263.22; 264/6

(57) ABSTRACT

Disclosed herein are methods of treating diseases and disorders responsive to inhibition of Hsp90, pharmaceutical compositions, pharmaceutical dosage forms and medicaments useful for the treatment of diseases responsive to inhibition of Hsp90, and methods of making the pharmaceutical compositions, pharmaceutical dosage forms and medicaments.

### RELATED APPLICATIONS

This application is a continuation of international patent application PCT/US2010/056522, filed Nov. 12, 2010, which claims the benefit of U.S. Provisional Application Ser. No. 61/261,258, filed Nov. 13, 2009; U.S. Provisional Application Ser. No. 61/285,882, filed Dec. 11, 2009; and U.S. Provisional Application Ser. No. 61/324,666, filed Apr. 15, 2010; the contents of all which are incorporated by reference herein in their entirety.

### FIELD OF THE INVENTION

The invention generally relates to the field of pharmaceutics for human therapy, and specifically to the development of methods of treating diseases, such as cancer, responsive to the inhibition of Hsp90, and pharmaceutical compositions and pharmaceutical dosage forms useful in such methods for the treatment of such diseases.

### BACKGROUND OF THE INVENTION

Cancer is prevalent: Among United States citizens that live to be 70 years old, the probability of developing invasive cancer is 38% for females and 46% for males. According to the American Cancer Society, there will be about 1.4 million new cases of cancer in the United States alone in 2006. Although the five year survival rate for all cancers is now 65%, up from about 50% in the mid-nineteen seventies, cancer remains a leading killer today. Indeed, it is estimated that 565,000 people in the United States will die from cancer in 2006. (American Cancer Society, Surveillance Research, 2006). Although numerous treatments are available for various cancers, the fact remains that many cancers remain incurable, untreatable, and/or become resistant to standard therapeutic regimens. Thus, there is a clear need for new cancer treatments employing new chemotherapeutic compounds.

Inhibitors of the molecular chaperone protein Hsp90 are being developed as one class of pharmacological weaponry in the anticancer chemotherapeutic arsenal. U.S. Pat. No. 7,595,401, issued on Sep. 29, 2009, which is hereby incorporated by reference in its entirety, discloses a number of Hsp90 inhibitors. Consequently, there is a clear need for methods of using such inhibitors and formulations comprising such inhibitors for the treatment of diseases and disorders, such as cancer, that respond favorably to the inhibition of Hsp90.

### BRIEF SUMMARY OF THE INVENTION

Among other things, the present invention relates to methods of treating diseases and disorders, such as cancer, that are responsive to the inhibition of Hsp90.

The present invention is based upon the discovery that (2S)-1-[4-(2-{6-amino-8-[(6-bromo-1,3-benzodioxol-5-yl)thio]-9H-purin-9-yl}ethyl)piperidin-1-yl]-1-oxopropan-2-ol (structurally shown below and hereinafter referred to as “Compound 1”) is orally bioavailable in mammals. Additionally, it has been discovered that

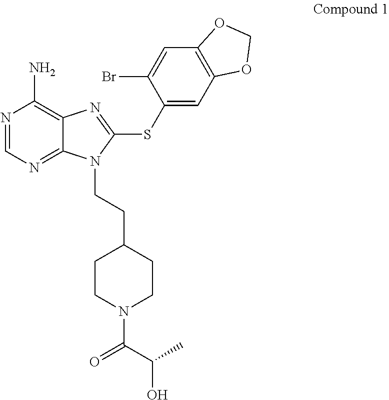

Compound 1 is efficacious in a wide variety of murine cancer xenograft models. Furthermore, it has been discovered that the pharmacokinetic properties and drug concentrations achievable in human patients administered Compound 1 orally are similar to those observed in efficacious murine cancer xenograft models. In view of these discoveries, the present invention comprises the following aspects

The present invention includes methods of treating or preventing diseases and disorders responsive to the inhibition of Hsp90 in a mammal, particularly a human patient, in need thereof.

In some embodiments, the method comprises orally administering to the mammal having an Hsp90 responsive disease or disorder, such as cancer, and particularly a human patient having such a disease or disorder, a therapeutically-effective amount of Compound 1, or a pharmaceutically-acceptable salt thereof.

In some embodiments, the method comprises administering to the mammal a therapeutically-effective amount of Compound 1, sufficient to provide in the mammal a plasma Cₘₐₓ of about 1,500 ng/mL to about 30,000 ng/mL of Compound 1, or an amount of a pharmaceutically-acceptable salt of Compound 1 sufficient to achieve an equimolar concentration in the plasma of the mammal.

In some embodiments, the method comprises administering to the mammal a therapeutically-effective amount of Compound 1 sufficient to provide in the mammal an AUC of about 10,000 hr*ng/mL to about 700,000 hr*ng/mL of Compound 1, or an amount of a pharmaceutically-acceptable salt of Compound 1 sufficient to achieve an equivalent exposure in the mammal. The AUC may be calculated over a 12 hour interval “AUC(0-12)”, over a 24 hour interval “AUC(0-24)”, or over an infinite time interval “AUC(0-inf)”.

In some of these embodiments, Compound 1, or a pharmaceutically-acceptable salt thereof, is administered orally as a solid pharmaceutical dosage form, such as a tablet. Thus, other aspects of the present invention include pharmaceutical compositions, pharmaceutical dosage forms and medicaments comprising Compound 1, or a pharmaceutically-acceptable salt thereof.

In some embodiments the pharmaceutical composition or medicament comprises Compound 1, or a pharmaceutically-acceptable salt thereof, and at least one pharmaceutically-acceptable solubilizing agent. In some embodiments the pharmaceutical composition comprises an amount of Compound 1 ranging from about 20 mg to about 200 mg, or an equivalent amount of a pharmaceutically-acceptable salt thereof.

In some embodiments, the pharmaceutical dosage form comprises a pharmaceutical composition of the present invention and at least one liquid pharmaceutically-acceptable carrier.

In some embodiments, the pharmaceutical dosage form comprises a pharmaceutical composition of the present invention and at least one pharmaceutically-acceptable excipient.

The present invention also encompasses a method of making pharmaceutical compositions, pharmaceutical dosage forms, and medicaments. The methods of making pharmaceutical compositions comprise mixing Compound 1, or a pharmaceutically-acceptable salt thereof, with at least one pharmaceutically-acceptable solubilizing agent. The methods of making pharmaceutical dosage forms and medicaments comprise mixing Compound 1, or a pharmaceutically-acceptable salt thereof, with at least one solubilizing agent to form a mixture, and mixing this mixture, or a pharmaceutical composition comprising Compound 1, or a pharmaceutically-acceptable salt thereof, with at least one pharmaceutically-acceptable excipients to create a pharmaceutical dosage form.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, drawings and claims.

### BRIEF DESCRIPTION OF THE DRAWINGS

FIG. 1 depicts the effects of Compound 1 and SNX-5422 on N-87 Her2+ gastric carcinoma xenografts in mice.

FIGS. 2A and 2B depict the human plasma pharmacokinetics of Compound 1.

FIG. 3 depicts Hsp70 levels in human patients treated with Compound 1.

FIG. 4 depicts tumor volume in xenografted mice dosed orally with Compound 1.

FIG. 5 depicts tumor volume in xenografted mice dosed orally with Compound 1 once-a-day and twice-a-day.

FIG. 6 depicts plasma concentration and liver Hsp70 RNA amounts in xenografted mice after oral dosing with Compound 1.

FIG. 7 depicts tumor volume in xenografted mice dosed orally with Compound 1 or erlotinib.

FIG. 8A depicts tumor volume in xenografted mice dosed orally with Compound 1 or intraperitoneally with 5-fluorouracil.

FIG. 8B depicts the time until tumor volume exceeded 1500 mm³ for the xenografted mice for which tumor volume results are depicted in FIG. 8A.

FIG. 9A depicts the plasma concentration of Compound 1 in female Sprague Dawley rats dosed orally once with Compound 1.

FIG. 9B depicts the plasma concentration of Compound 1 in female Sprague Dawley rats dosed orally twice, twelve hours apart, with Compound 1.

FIG. 10 depicts an overview of a process, according to embodiments of the invention, used for making solid pharmaceutical dosage forms comprising Compound 1.

FIG. 11 depicts an overview of another process, according to some embodiments of the invention, used for making solid pharmaceutical dosage forms comprising Compound 1.

### DETAILED DESCRIPTION OF THE INVENTION

The present invention relates to methods of treating diseases and disorders responsive to the inhibition of Hsp90, such as cancer, in mammals, and particularly in human patients, and to pharmaceutical compositions, pharmaceutical dosage forms and medicaments useful in such methods of treatment.

The present invention is based upon the discovery that (2S)-1-[4-(2-{6-amino-8-[(6-bromo-1,3-benzodioxol-5-yl)thio]-9H-purin-9-yl}ethyl)piperidin-1-yl]-1-oxopropan-2-ol (structurally shown below and hereinafter referred to as “Compound 1”) is orally bioavailable in mammals. Additionally, it has been discovered that

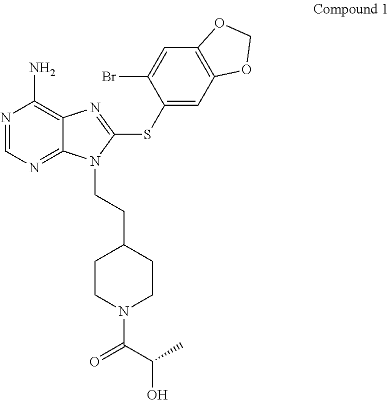

Compound 1 is efficacious in a wide variety of murine cancer xenograft models. Furthermore, it has been discovered that the pharmacokinetic properties and drug concentrations achievable in human patients administered Compound 1 orally are similar to those observed in efficacious murine cancer xenograft models. In view of these discoveries, the present invention comprises the following aspects.

The present invention includes and provides methods of treating or preventing diseases and disorders responsive to the inhibition of Hsp90, such as cancer, in a mammal in need thereof.

In some embodiments, the method comprises orally administering to a mammal (e.g., a human patient) having an Hsp90 responsive disease or disorder, such as cancer, a therapeutically-effective amount of Compound 1, or a pharmaceutically-acceptable salt thereof.

A wide variety of cancers are likely to be responsive to Hsp90 inhibition. Without wishing to be bound by theory, the molecular chaperone heat shock protein 90 (Hsp90) plays a role in stabilizing and activating hundreds of proteins—so-called client proteins—many of which participate in cell signaling and stress response pathways. Tumor cells are especially reliant on Hsp90, because of its function in assisting in the folding of a number of overexpressed and mutant proteins. These oncoproteins support features unique to cancer cells, such as excessive proliferation and inappropriate survival (Trepel et al. Nat. Rev. Cancer. 10(8):537, 2010). Thus, a wide variety of cancers are likely to be responsive to Hsp90 inhibition.

For example, one such oncoprotein, the growth factor receptor HER2, is overexpressed in roughly one quarter of breast cancers (HER2-positive breast cancer) and drives progression of this tumor type. The HER2 protein is very sensitive to inhibition of Hsp90, and forms the basis for the exploration of HER2-positive breast cancer treatment with Hsp90 inhibitors (Mimnaugh et al., J. Biol. Chem. 271:22796, 1996).

In non-small cell lung cancer, the epidermal growth factor receptor (EGFR) plays a central role in driving tumor growth. Patients on EGFR inhibitor therapy can have tumor progression due to oncogenic switching, wherein tumors become less dependent on EGFR and more dependent on alternative growth factor receptors, such as HER2, BRAF, MET, and ALK. These alternative receptors are all Hsp90 clients, and combined EGFR/Hsp90 inhibitor treatment can block this switch (Sequist et al., J. Clin. Oncol. Abstr. 27, 8073, 2009).

In multiple myeloma tumor cells, Hsp90 inhibition completely abrogates cell surface expression of two important growth factor receptors: insulin-like growth factor receptor and interleukin-6 receptor (Mitsaides et al., Blood 107(3):1092, 2006). In addition, the G-protein coupled receptor 6, a myeloma survival kinase, has also been characterized as an Hsp90 client protein (Tiedemann et al. Blood 115(8):1594, 2010).

In acute myelogenous leukemia (AML), the FLT3 growth factor receptor is frequently mutated and constitutively activated, driving tumor progression. In chronic myelogenous leukemia (CML), tumors are characterized by the common BCR-ABL fusion protein. Both mutant FLT3 and BCR-ABL proteins are Hsp90 clients. Therefore, both AML and CML tumors may be responsive to Hsp90 inhibition.

Additionally, the Janus kinase 2 (JAK2) protein has been shown to be an Hsp90 client protein. JAK2 mutations are common in myeloproliferative disorders such as polycythemia vera, essential thromocytosis, and primary myelofibrosis, and Hsp90 inhibition has been shown to have anti-tumor activity in JAK2-dependent models of malignancy (Marubayashi et al., J. Clin. Invest. 120(10):3578, 2010).

Frequently, tumors ultimately develop resistance to kinase inhibitor therapy by the occurrence of mutations within targeted oncogenic kinases, which block binding of kinase inhibitors. Hsp90 inhibitors have been shown to overcome such primary resistance mutations in CML (Gorre et al., Blood 100(8):3041, 2007), GIST (Bauer et al., Cancer Res. 66(18):9153, 2006), and NSCLC (Shimamura et al., Cancer Res. 68(14):5827, 2008).

In some embodiments, the cancer to be treated is selected from, but is not limited to, Hodgkin's disease, non-Hodgkin's lymphoma, acute lymphocytic leukemia, chronic lymphocytic leukemia, multiple myeloma, acute myelogenous leukemia, chronic myelogenous leukemia, myeloproliferative neoplasms, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, non-small cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, primary myelofibrosis, adrenal cortex carcinoma, skin cancer, prostatic carcinoma, and combinations thereof.

In some embodiments, the cancer comprises gastric cancer, colon cancer, prostate cancer, small-cell lung cancer, non-small cell lung cancer, ovarian cancer, acute myeloid leukemia, multiple myeloma, renal cell carcinoma, gastrointestinal stromal tumor, chronic myeloid leukemia, glioblastoma multiforme, astrocytomas, medulloblastomas, melanoma, breast cancer, pancreatic cancer, and combinations thereof.

In other embodiments, the diseases to be treated or prevented comprise viral infections, such as, for example, hepatitis B and C viruses, HIV, herpes viruses, SARS coronavirus, and influenza viruses.

In other embodiments, the diseases and disorders to be treated or prevented comprise neurodegenerative diseases and disorders, such as, for example, Alzheimer's disease, other tautopathies (such as fronto-temporal dementia, progressive supranuclear palsy, and corticobasal degeneration), spinal and bulbar muscular atrophy, Huntington's disease (Huntingtin aggregates), Parkinson's disease (alpha-synuclein aggregates), stroke (ischemic stress), autoimmune encephalomyelitis, spinocerebellar ataxia, transmissible spongiform encephalopathies (prion misfolding), and demylelinating neuropathies.

In still yet other embodiments, the diseases and disorders to be treated or prevented comprise inflammation diseases and disorders, such as, for example, multiple sclerosis (antibody-mediated), inflammatory bowel disease, gastritis, arthritis, and uveitis.

In further embodiments, the diseases and disorders to be treated or prevented comprise fungal diseases, graft-versus-host disease, and parasitic diseases, such as, for example, malaria, toxoplasmosis, trypanosomiasis, and leishmaniasis.

In some embodiments, the method comprises administering to the mammal, and particularly a human patient, a therapeutically-effective amount of Compound 1 sufficient to provide in the mammal or human patient a plasma Cₘₐₓ of about 1,500 ng/mL to about 30,000 ng/mL, or an amount of a pharmaceutically-acceptable salt of Compound 1 sufficient to achieve an equimolar concentration in the plasma of the mammal or human patient. In some of such embodiments, Compound 1 is administered orally. In such embodiments, administering Compound 1 comprises administering any of the pharmaceutical compositions, pharmaceutical dosage forms, or medicaments disclosed herein, or any similar pharmaceutical composition, pharmaceutical dosage form, or medicament comprising a therapeutically-effective amount of Compound 1. In such embodiments, administering to the mammal, and particularly a human patient, a therapeutically-effective amount of Compound 1 comprises administering the pharmaceutical composition, pharmaceutical dosage form, or medicament comprising a therapeutically-effective amount of Compound 1 once-a-day, two-times-a-day (i.e., twice daily), three-times-a-day, or four-times-a-day.

In particular embodiments, the Cₘₐₓ of Compound 1 to be achieved with daily dosing ranges from about 6,000 ng/mL to about 30,000 ng/mL.

In particular embodiments, the Cₘₐₓ of Compound 1 to be achieved with twice daily dosing ranges from about 6,000 ng/mL to about 15,000 ng/mL.

In some embodiments, the method comprises administering to the mammal, and particularly a human patient, a therapeutically-effective amount of Compound 1 sufficient to provide in the mammal or human patient an AUC ranging from about 10,000 hr*ng/mL to about 700,000 hr*ng/mL, or administering an amount of a pharmaceutically-acceptable salt of Compound 1 sufficient to achieve an equivalent exposure in the mammal or human patient. The AUC may be calculated over a 12 hour interval “AUC(0-12)”, over a 24 hour interval “AUC(0-24)”, or over an infinite time interval “AUC(0-inf)”. In some of such embodiments, Compound 1 is administered orally. In some of such embodiments, administering Compound 1 comprises administering any of the pharmaceutical compositions or pharmaceutical dosage forms disclosed herein, or any similar pharmaceutical composition, pharmaceutical dosage form, or medicament comprising a therapeutically-effective amount of Compound 1. In some of such embodiments, administering to the mammal, and particularly the human patient, a therapeutically-effective amount of Compound 1 comprises administering pharmaceutical composition, pharmaceutical dosage form, or medicament comprising a therapeutically-effective amount of Compound 1, two times a day.

In some embodiments, the AUC(0-24) of Compound 1 to be achieved with a daily dose ranges from about 90,000 hr*ng/mL to about 400,000 hr*ng/mL.

In some embodiments, the AUC(0-inf) of Compound 1 to be achieved with a daily dose ranges from about 130,000 hr*ng/mL of Compound 1 to about 700,000 hr*ng/mL.

In some embodiments, the AUC(0-12) of Compound 1 to be achieved with a twice daily dose ranges from about 30,000 hr*ng/mL to about 80,000 hr*ng/mL.

In some embodiments, the AUC(0-inf) of Compound 1 to be achieved with a twice daily dose ranges from about 50,000 hr*ng/mL to about 300,000 hr*ng/mL. In some embodiments, the AUC(0-inf) of Compound 1 to be achieved with a twice daily dose ranges from about 50,000 hr*ng/mL to about 200,000 hr*ng/mL.

In some embodiments, the therapeutically-effective amount of Compound 1, or an equimolar amount of a pharmaceutically-acceptable salt thereof, ranges from about 50 mg/m² to about 600 mg/m². As used herein, “mg/m²” refers to the dosage in mg of Compound 1 per square meter of body surface area of the recipient. It should be clear to the skilled artisan that if a pharmaceutically-acceptable salt of Compound 1 is being administered, then the dosage is to be scaled accordingly to administer an equivalent dosage (i.e., equimolar amount) of the pharmaceutically-acceptable salt Compound 1.

In some embodiments, the therapeutically-effective amount of Compound 1 to be administered, or equimolar amount of a pharmaceutically-acceptable salt thereof, is about 50 mg/m², about 100 mg/m², about 150 mg/m², about 200 mg/m², about 250 mg/m², about 300 mg/m², about 350 mg/m², about 400 mg/m², about 450 mg/m², about 500 mg/m², about 550 mg/m², or about 600 mg/m², per day.

In some embodiments, the therapeutically-effective amount of Compound 1, or an equimolar amount of a pharmaceutically-acceptable salt thereof, is greater than about 600 mg/m² per day.

In some embodiments, the therapeutically-effective amount of Compound 1, or an equimolar amount of a pharmaceutically-acceptable salt thereof, is about 50 mg/m² per day.

In some embodiments, the therapeutically-effective amount of Compound 1, or an equimolar amount of a pharmaceutically-acceptable salt thereof, is about 100 mg/m² per day.

In some embodiments, the therapeutically-effective amount of Compound 1, or an equimolar amount of a pharmaceutically-acceptable salt thereof, is about 165 mg/m² per day.

In some embodiments, the therapeutically-effective amount of Compound 1, or an equimolar amount of a pharmaceutically-acceptable salt thereof, is about 176 mg/m² per day.

In some embodiments, the therapeutically-effective amount of Compound 1, or an equimolar amount of a pharmaceutically-acceptable salt thereof, is about 245 mg/m² per day.

In some embodiments, the therapeutically-effective amount of Compound 1, or an equimolar amount of a pharmaceutically-acceptable salt thereof, is about 340 mg/m² per day.

In some embodiments, the therapeutically-effective amount of Compound 1, or an equimolar amount of a pharmaceutically-acceptable salt thereof, ranges from about 25 to about 600 mg/m², twice a day.

In some embodiments, the therapeutically-effective amount of Compound 1, or an equimolar amount of a pharmaceutically-acceptable salt thereof, is about 25 mg/m², about 50 mg/m², about 75 mg/m², about 100 mg/m², about 150 mg/m², about 200 mg/m², about 250 mg/m², about 300 mg/m², about 350 mg/m², about 400 mg/m², about 450 mg/m², about 500 mg/m², about 550 mg/m², or about 600 mg/m², twice a day.

In some embodiments, the therapeutically-effective amount of Compound 1, or an equimolar amount of a pharmaceutically-acceptable salt thereof, is greater than about 600 mg/m² twice a day.

In some embodiments, the therapeutically-effective amount of Compound 1, or an equimolar amount of a pharmaceutically-acceptable salt thereof, is about 25 mg/m² twice a day.

In some embodiments, the therapeutically-effective amount of Compound 1, or an equimolar amount of a pharmaceutically-acceptable salt thereof, is about 50 mg/m² twice a day.

In some embodiments, the therapeutically-effective amount of Compound 1, or an equimolar amount of a pharmaceutically-acceptable salt thereof, is about 100 mg/m² twice a day.

In some embodiments, the therapeutically-effective amount of Compound 1, or an equimolar amount of a pharmaceutically-acceptable salt thereof, is about 165 mg/m² twice a day.

In some embodiments, the therapeutically-effective amount of Compound 1, or an equimolar amount of a pharmaceutically-acceptable salt thereof, is about 176 mg/m² twice a day.

In some embodiments, the therapeutically-effective amount of Compound 1, or an equimolar amount of a pharmaceutically-acceptable salt thereof, is about 245 mg/m² twice a day.

In some embodiments, the therapeutically-effective amount of Compound 1, or an equimolar amount of a pharmaceutically-acceptable salt thereof, is about 340 mg/m² twice a day.

In some embodiments, the therapeutically-effective amount of Compound 1, or an equimolar amount of a pharmaceutically-acceptable salt thereof, ranges from about 100 mg to about 1000 mg, per day.

In some embodiments, the therapeutically-effective amount of Compound 1, or an equimolar amount of a pharmaceutically-acceptable salt thereof, is about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, per day.

In some embodiments, the therapeutically-effective amount of Compound 1, or an equimolar amount of a pharmaceutically-acceptable salt thereof, is greater than about 1000 mg per day.

In some embodiments, the therapeutically-effective amount of Compound 1, or an equimolar amount of a pharmaceutically-acceptable salt thereof, is about 100 mg per day.

In some embodiments, the therapeutically-effective amount of Compound 1, or an equimolar amount of a pharmaceutically-acceptable salt thereof, is about 160 mg per day.

In some embodiments, the therapeutically-effective amount of Compound 1, or an equimolar amount of a pharmaceutically-acceptable salt thereof, ranges from about 340 mg to about 540 mg, per day.

In some embodiments, the therapeutically-effective amount of Compound 1, or an equimolar amount of a pharmaceutically-acceptable salt thereof, ranges from about 480 mg to about 620 mg, per day.

In some embodiments, the therapeutically-effective amount of Compound 1, or an equimolar amount of a pharmaceutically-acceptable salt thereof, ranges from about 540 mg to about 740 mg, per day.

In some embodiments, the therapeutically-effective amount of Compound 1, or an equimolar amount of a pharmaceutically-acceptable salt thereof, ranges from about 480 mg, per day.

In some embodiments, the therapeutically-effective amount of Compound 1, or an equimolar amount of a pharmaceutically-acceptable salt thereof, ranges from about 25 mg to about 1000 mg, twice per day.

In some embodiments, the therapeutically-effective amount of Compound 1, or an equimolar amount of a pharmaceutically-acceptable salt thereof, is about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, twice per day.

In some embodiments, the therapeutically-effective amount of Compound 1, or an equimolar amount of a pharmaceutically-acceptable salt thereof, is greater than about 1000 mg twice per day.

In some embodiments, the therapeutically-effective amount of Compound 1, or an equimolar amount of a pharmaceutically-acceptable salt thereof, is about 25 mg twice per day.

In some embodiments, the therapeutically-effective amount of Compound 1, or an equimolar amount of a pharmaceutically-acceptable salt thereof, is about 50 mg twice per day.

In some embodiments, the therapeutically-effective amount of Compound 1, or an equimolar amount of a pharmaceutically-acceptable salt thereof, is about 75 mg twice per day.

In some embodiments, the therapeutically-effective amount of Compound 1, or an equimolar amount of a pharmaceutically-acceptable salt thereof, is about 100 mg twice per day.

In some embodiments, the therapeutically-effective amount of Compound 1, or an equimolar amount of a pharmaceutically-acceptable salt thereof, is about 165 mg twice per day.

In some embodiments, the therapeutically-effective amount of Compound 1, or an equimolar amount of a pharmaceutically-acceptable salt thereof, is about 240 mg twice per day.

In some embodiments, the therapeutically-effective amount of Compound 1, or an equimolar amount of a pharmaceutically-acceptable salt thereof, ranges from about 270 to about 370 mg, twice per day.

In some embodiments, the therapeutically-effective amount of Compound 1, or an equimolar amount of a pharmaceutically-acceptable salt thereof, is about 500 mg twice per day.

In some embodiments, administration of Compound 1, or a pharmaceutically-acceptable salt thereof, results in at least about a 50% regression in tumor volume.

In some embodiments, administration of Compound 1, or a pharmaceutically-acceptable salt thereof, results in at least about a 50% inhibition of tumor growth.

In some embodiments, administration of Compound 1, or a pharmaceutically-acceptable salt thereof, results in inhibition of tumor growth ranging from at least about 50% inhibition to about 50% regression in tumor volume.

In some embodiments, administration of Compound 1, or a pharmaceutically-acceptable salt thereof, results in at least about 10%, about 20%, about 30%, about 40%, about 50%, about 60%, about 70%, about 80%, about 90%, or about 100% inhibition of tumor growth.

In some embodiments, administration of Compound 1, or a pharmaceutically-acceptable salt thereof, results in at about 10%, about 20%, about 30%, about 40%, about 50%, about 60%, about 70%, about 80%, about 90%, or about 100% regression in tumor volume.

In some embodiments, Compound 1 has improved safety characteristics as compared to other Hsp90 inhibitors, such as, for example, SNX-5422.

In some embodiments, the method further comprises determining the effect of the administration step by monitoring Hsp90 inhibition in the mammal or human patient. In some of these embodiments, the monitoring step comprises monitoring Hsp70 levels in peripheral blood mononuclear cells, wherein an increase in Hsp70 level indicates Hsp90 inhibition.

In some embodiments, the mammal is a human patient in need of such treatment who is identified as being in need of such treatment by way of a diagnostic assay. In particular embodiments the diagnostic assay used to identify a human patient as being in need of such treatment is conducted on a biological sample, such as a biopsy sample, containing abnormal, diseased or cancerous cells, that is obtained from a candidate patient.

In other embodiments, the mammal is a human patient in need of such treatment who is identified as being in need of such treatment by way of an efficacy assay conducted on abnormal, diseased or cancerous cells, obtained from a sample, such as a biopsy, removed from a candidate patient.

In addition to the above methods, the present invention also relates specifically to the development of pharmaceutical compositions and pharmaceutical dosage forms useful for the treatment of diseases responsive to inhibition of Hsp90 and to methods related thereto.

As used herein, the term “dose” or “dosage” refers to the amount of active pharmaceutical ingredient that an individual takes or is administered at one time. For example, an 50 mg dose of Compound 1 refers to, in the case of a twice-daily dosage regimen, a situation where, for example, the individual takes, or is administered, 50 mg of Compound 1 in the morning and 50 mg of Compound 1 in the evening. The 50 mg Compound 1 dose can be administered in a single dosage unit or can be divided into two or more dosage units, e.g., two 25 mg Compound 1 dosage units.

As used herein, the term “pharmaceutical dosage form or dosage unit” refers to a physically discrete unit, such as a tablet, capsule, or sachet containing a unitary dosage for a human patient. Each pharmaceutical dosage form or dosage unit contains a predetermined quantity of Compound 1.

The term “excipient,” as used herein, refers to those components of a pharmaceutical composition or pharmaceutical dosage form, other than Compound 1, that are intentionally included in the composition or formulation to either facilitate manufacture, enhance stability, control the release of Compound 1 from the drug product, assist in product identification, or enhance any other product characteristics, including, for example, the pharmacokinetics of the drug product. Generally, excipients may be thought of as the “inactive ingredients” of the pharmaceutical composition or pharmaceutical dosage form, in the sense that they exert no direct therapeutic effect. However, excipients can have an effect on the pharmacokinetic characteristics of the active pharmaceutical ingredient (i.e., Compound 1) in pharmaceutical compositions or pharmaceutical dosage forms comprising them. For example, different excipients, or combinations of excipients, can alter the dissolution rate of tablets, and thereby alter the pharmacokinetic characteristics of the active pharmaceutical ingredient contained in the tablet.

As used herein, the term “pharmaceutical dosage form,” is used to refer to a finished pharmaceutical product or medicament that is suitable for administration to a mammal, or a human patient. The pharmaceutical dosage form can be thought of as comprising a pharmaceutical composition in combination with one or more excipients or carriers.

It has been discovered that the bioavailability of Compound 1 is improved when Compound 1 is formulated with a solubilizing agent. Thus, in some embodiments of the pharmaceutical composition, the pharmaceutical composition comprises Compound 1, or a pharmaceutically-acceptable salt thereof, and at least one pharmaceutically-acceptable solubilizing agent.

In some embodiments, the at least one pharmaceutically-acceptable solubilizing agent comprises a pharmaceutically-acceptable cyclodextrin. In some embodiments, the pharmaceutically-acceptable cyclodextrin comprises a beta-cyclodextrin. In some of these embodiments, the pharmaceutically-acceptable cyclodextrin comprises a hydroxypropyl beta-cyclodextrin (HPbCD), such as, for example, Encapsin® (RDI, Concord, Mass.). In others of these embodiments, the pharmaceutically-acceptable beta-cyclodextrin comprises sulfobutylether beta-cyclodextrin (SBEbCD). Non-limiting examples of SBEbCD include Captisol® (CyDex Pharmaceuticals, Inc.; Lenexa, Kans.) with an average molecular weight of 2160 Daltons and an average degree of substitution of 7.

In some embodiments, the molar ratio of the pharmaceutically-acceptable cyclodextrin, such as SBEbCD, to Compound 1, or a pharmaceutically-acceptable salt thereof, ranges from about 1:1 to about 6:1 moles of SBEbCD to moles of Compound 1 (i.e., from about 1 to about 6 molecules of SBEbCD per molecule of Compound 1). In some of these embodiments, the molar ratio of the pharmaceutically-acceptable cyclodextrin, such as SBEbCD, to Compound 1 is about 3:1 (i.e., about 3 molecules of cyclodextrin, such as SBEbCD, per molecule of Compound 1).

In some embodiments of the pharmaceutical composition, the pharmaceutical composition comprises and amount of Compound 1 that ranges from about 20 mg to about 200 mg, or an equivalent amount of a pharmaceutically-acceptable salt of Compound 1.

In some embodiments, the pharmaceutical composition comprises about 20 mg, about 40 mg, about 60 mg, about 80 mg, about 100 mg, about 120 mg, about 140 mg, about 160 mg, about 180 mg, or about 200 mg of Compound 1, or an equivalent amount of a pharmaceutically-acceptable salt of Compound 1.

In some embodiments, the pharmaceutical dosage form comprises the herein disclosed pharmaceutical composition and at least one liquid pharmaceutically-acceptable carrier, thereby forming a liquid pharmaceutical dosage form. In some of these embodiments, the at least one liquid pharmaceutically-acceptable carrier comprises water, a dextrose solution, normal saline, or normal saline plus dextrose. In other embodiments, the at least one liquid pharmaceutically-acceptable carrier comprises water and at least one other pharmaceutically-acceptable ingredient in an aqueous mixture. In some of these embodiments, the aqueous mixture is a homogeneous mixture (i.e., a solution). In some of these embodiments, the aqueous mixture is a heterogeneous mixture. In some embodiments, the mixture has an acidic pH.

In some embodiments of the pharmaceutical dosage form, the pharmaceutical dosage form comprises the herein disclosed pharmaceutical composition and at least one solid pharmaceutically-acceptable excipient, thereby forming a solid pharmaceutical dosage form. In some of these embodiments, the solid pharmaceutical dosage form is formulated as granules, capsules containing granules, uncoated tablets, or coated tablets. In some of these embodiments, the solid pharmaceutical dosage form comprises an amount of Compound 1 that ranges from about 20 mg to about 200 mg, or an equivalent amount of a pharmaceutically-acceptable salt of Compound 1. In some of these embodiments, the solid pharmaceutical dosage form is uncoated or coated tablets comprising an amount of Compound 1 that ranges from about 20 mg to about 75 mg, or an equivalent amount of a pharmaceutically-acceptable salt of Compound 1.

In some embodiments, the at least one solid pharmaceutically-acceptable excipient comprises at least one binder, at least one diluent, at least one tableting agent, at least one flavoring agent, at least one sweetening agent, at least one coating agent, or combinations thereof.

Aspects of the present invention also provide methods of making a solid pharmaceutical dosage form comprising Compound 1, or a pharmaceutically-acceptable salt thereof. These methods comprise mixing Compound 1, or the pharmaceutically-acceptable salt thereof, with at least one solubilizing agent to form a mixture.

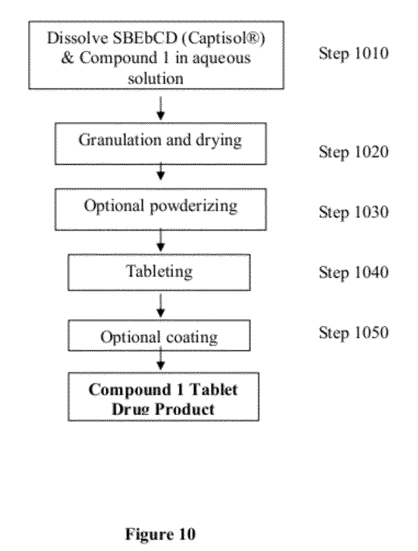

In some embodiments, the at least one solubilizing agent comprises a pharmaceutically-acceptable cyclodextrin. In some of these embodiments, the mixing comprises dissolving Compound 1, or a pharmaceutically-acceptable salt thereof, and said pharmaceutically-acceptable cyclodextrin an aqueous mixture to form a solution that can serve as a granulation medium. See, e.g., Step 1010 of FIG. 10.

In some embodiments of the methods of making a pharmaceutical dosage form, the methods further comprises forming granules in a granulation process using the granulation medium and at least one binder and at least one diluent. See, e.g., Step 1020 of FIG. 10.

Any granulation process known in the art may be used. For example, in some embodiments, the granulation process comprises using a fluid bed process. In another example, in some embodiments, the granulation process comprises using a high shear granulation process. Such granulation processes, and the equipment involved in carrying out the processes, are known in the art and within the purview of the skilled artisan.

In some embodiments, the at least one diluent employed in the granulation process comprises microcrystalline cellulose. In some embodiments, the at least one diluent comprises microcrystalline cellulose, mannitol, hydrous or anhydrous lactose, sucrose, sorbitol, dicalcium phosphate, or combinations thereof.

In some embodiments, the at least one binder employed in the granulation process comprises hypromellose. In some embodiments, the at least one binder comprises hypromellose, polyvinylpyrrolidone, starch, or combination thereof.

In some embodiments of the granulation process, the method further comprises using at least one flavoring agent and/or sweetening agent in addition to the at least one binder and at least one diluent.

In some embodiments of the granulation process, the method further comprises optionally including additives such as preservatives to inhibit or prevent microbial growth, and/or antioxidants or other chemical stabilizers. The preservatives, antioxidants, or chemical stabilizers may be added directly to the granulation medium during its preparation, or subsequent to the preparation of the granulation medium.

In some embodiments of the method of making a pharmaceutical dosage form, the method further comprises forming powder from the granules in a powderizing process. See, e.g., Step 1030 of FIG. 10. In some of these embodiments, the powderizing process comprises milling, grinding, or pulverizing the granules.

In some embodiments of the method of making a pharmaceutical dosage form, the method further comprises forming tablets in a tableting process using the powder formed by the powderizing process and at least one tableting agent. In other embodiments of the method of making a pharmaceutical dosage form, the powderizing process is bypassed and the method further comprises forming tablets in a tableting process using the granules and at least one tableting agent. See, e.g., Step 1040 of FIG. 10.

In some embodiments of the method of making a pharmaceutical dosage form, the at least one tableting agent is at least one diluent, at least one compression aid, at least one disintegrant, at least one glidant, at least one lubricant, or a combination thereof. In some of these embodiments, the at least one tableting agent comprises microcrystalline cellulose, croscarmellose sodium, crospovidone, colloidal silicon dioxide, magnesium stearate, mannitol, hydrous or anhydrous lactose, sucrose, sorbitol, dicalcium phosphate, or combinations thereof.

In some embodiments of the method of making a pharmaceutical dosage form when the pharmaceutical dosage form comprises a tablet, the method further comprises packaging the tablets in a suitable container. In some embodiments, the container comprises blister packs or bottles.

In some embodiments, where the pharmaceutical dosage form is a tablet, prior to packaging the tablets, the method further comprises film coating the tablets with at least one coating agent. See, e.g., Step 1050 of FIG. 10. In some embodiments, at least one coating agent comprises a cosmetic coating agent, sustained-release coating agent, controlled-release coating agent, enteric coating agent, or combination thereof. Some of these embodiments further comprise packaging the film-coated tablets in a suitable container, for example blister packs or bottles.

In some embodiments, instead of processing the granulation medium into granules, the method further comprises sterile filtration of the liquid granulation medium to form a sterile solution. In some embodiments, the method further comprises packaging the sterile solution in a suitable container (e.g., a vial). Optionally, the sterile-filtered granulation medium is filled into vials and the water removed via lyophilization or freeze-drying techniques to create a reconstitutable solid. In such embodiments the sterile-filtered granulation medium is suitable for either parenteral of oral administration.

In some embodiments, the method further comprises packaging the granulation medium solution in a container suitable for oral administration (e.g., a vial or bottle). In some embodiments, the method further comprises adding at least one flavoring and or sweetening agent to the granulation medium solution. In some embodiments, the at least one flavoring and or sweetening agent is added before packaging of the granulation medium solution is complete. In other embodiments, the at least one flavoring and or sweetening agent is added after the packaging has been opened, but prior to oral administration of the pharmaceutical dosage form. Optionally, the granulation medium is filled into vials and the water removed via lyophilization or freeze-drying techniques to create a reconstitutable solid.

In some embodiments, instead of using the granules to form tablets, the method further comprises filling capsules (e.g., hard gelatin capsules) with the granules.

In some embodiments of the method of forming granules, instead of using the granules to form tablets, the method further comprises packaging the granules in a container suitable for orally administering the granules. In some of these embodiments, the container comprises a bottle, capped glass or plastic vial, a laminated foil tear-open pouch, or a sealed cup with pull-off laminated foil lid. In some of these embodiments, the method further comprises adding water to the granules, mixing, and then orally administering the aqueous mixture to a subject. In some of these embodiments, the method further comprises adding a flavored liquid to the granules, mixing, and then orally administering the aqueous mixture to a subject. In these embodiments the flavored liquid comprises acidic liquids such as fruit juices or carbonated soft drinks. In other embodiments, the granules are intended to be sprinkled directly onto substrate carrier foods, such as, for example, applesauce, yogurt, or oatmeal where the granules are then orally ingested as the substrate carrier foods are consumed.

Non-limiting examples of excipients and carriers that can be used in these embodiments include binders, diluents, glidants, lubricants, disintegrating agents, granulating agents, tableting agents, flavoring agents, flavor-masking agents, sweeteners, stabilizers, preservative, coloring agents, and coating agents, and various combinations thereof.

Non-limiting examples of binders (substances that bind together Compound 1 to other carriers and excipients) include: acacia, alginates, such as sodium alginate or alginic acid, carbomers, carrageenan, cellulose derivatives, such as methyl cellulose (all grades/molecular chain lengths and viscosities), carboxymethylcellulose sodium, hydroxypropyl methyl cellulose (HPMC; hypromellose; Methocel® (Dow, Midland, Mich.)) (all grades/molecular chain lengths and viscosities), hydroxypropyl cellulose (HPC), hydroxyethyl cellulose, hydroxypropyl cellulose (e.g., Klucel® (Ashland, Wilmington, Del.)), polyvinylypyrollidone (PVP; all grades & chain lengths), copovidone (vinylpyrrolidone-vinyl acetate copolymer), hydroxyethylmethyl cellulose, cellulose acetate phthalate, ceratonia, chitosan, sucrose, corn syrup solids, dextrates, dextrin, dextrose, ethylcellulose, gelatin, glucose, sorbitol, glyceryl behenate, natural gums such as guar gum, hydrogenated vegetable oil, magnesium aluminum silicate, maltodextrin, maltose, methylcellulose, microcrystalline cellulose, poloxamer, polydextrose, polyethylene oxide, polyvinylpyrrolidone (e.g., povidone, Kollidon® (BASF, Germany), Plasdone® (ISP, Wayne, N.J.)), polyethylene glycols, starches (corn wheat, potato, rice), including pre-gelatinized starch (such as Starch 1500), sucrose, aqueous polymeric dispersions of ethyl cellulose (e.g., Aquacoat or SureRelease), and acrylate/methacrylate polymers and copolymers (e.g., Eudragits).

Non-limiting examples of disintegrants (substances that promote disintegration of a pharmaceutical dosage form) include: alginic acid and sodium alginate, guar gum, carboxymethyl cellulose calcium, carboxymethyl cellulose sodium, croscarmellose sodium (Ac-Di-Sol), crosslinked polyvinylpyrrolidone, crospovidone, powdered cellulose, chitosan, sodium starch glycolate (Explotab, Primojel), starch and pre-gelatinized starch, magnesium aluminum silicate, methylcellulose, and microcrystalline cellulose (all grades).

Non-limiting examples of diluents include: calcium carbonate, calcium phosphate, calcium sulfate, cellulose, cellulose acetate, compressible sugar, confectioner's sugar, dextrates, dextrin, dextrose, ethyl cellulose, fructose, fumaric acid, glyceryl palmitostearate, hydrogenated vegetable oil, kaolin, lactitol, lactose, magnesium carbonate, magnesium oxide, maltodextrin, maltose, mannitol, microcrystalline cellulose, polydextrose, polymethylacrylates, simethicone, sodium alginate, sodium chloride, sorbitol, starch, pregelantized starch, sterilizable maize, sucrose, sugar spheres, talc, tragacanth, trehalose, and xylitol.

Non-limiting examples of flavoring agents, flavor-masking agents, and sweeteners include: acesulfame potassium, aspartame, citric acid, dibutyl sebacate, ethyl maltol, fructose, maltol, monosodium glutamate, saccharin, saccharin sodium, sodium cyclamate, tartaric acid, trehalose, xylitol, sugar (sucrose), ethyl maltol, ethyl vanillin, fumaric acid, malic acid, maltol, menthol, phosphoric acid, triethyl citrate, and vanillin.

Non-limiting examples of lubricants include: magnesium stearate, sodium stearyl fumarate (e.g., PRUV), calcium stearate, magnesium lauryl sulfate, medium-chain triglycerides, polyethylene glycol (molecular weight 6000 and above), sodium lauryl sulfate, stearic acid, zinc stearate, and talc.

Non-limiting examples of preservatives, antioxidants, and chemical stabilizers include: ethanol, benzalkonium chloride, benzethonium chloride, benzyl alcohol, butylated hydroxyanisole (BHA), butylated hydroxytoluene (BHT), butylparaben, methylparaben, ethylparaben, propyl paraben, chlorbutanol, chlorhexidine, hexetidine, isopropyl alcohol, monothioglycerol, phenol, phenoxyethanol, phenylethyl alcohol, phenylmercuric acetate, phenylmercuric borate, phenylmercuric nitrate, potassium benzoate, potassium metabisulfite, potassium sorbate, propylene glycol, propyl gallate, sodium benzoate, sodium metabisulfite, sodium propionate, sorbic acid, ascorbic acid, and thimerosal.

Non-limiting examples of glidants and powder flow aids include: colloidal silicone dioxide, silicon dioxide, calcium phosphate, calcium silicate, powdered cellulose, magnesium silicate, magnesium trisilicate, starch, and talc.

Non-limiting examples of coatings for tablets include: hydroxypropyl methylcellulose, polyvinyl alcohol, Opadry® and Opadry® II (Colorcon, Harleysville, Pa.) coating systems, cellulose ethers and cellulose esters, polyacrylates, polymethacrylates, cellulose acetate phthalate, and polyvinyl acetate phthalate.

Methods of synthesizing Compound 1, and also potential pharmaceutically-acceptable salts of Compound 1, are disclosed in U.S. Pat. No. 7,595,401, issued on Sep. 29, 2009, the contents of which are incorporated by reference herein their entirety. Other methods of synthesizing Compound 1, and related compounds, are disclosed in International Patent Application PCT/US2008/083636, filed Nov. 14, 2008, and published as WO/2009/065035, on May 22, 2009, the contents of which are incorporated by reference herein their entirety.

Additionally, in Compound 1, any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039, which are incorporated by reference herein their entirety. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibits a significantly longer half-life in the bodies of patients administered such compounds. This particularly so when the catabolism of the hydrogenated compound is mediated by cytochrome P450 systems. See Kushner et al., Can. J. Physiol. Pharmacol. 77:79-88, 1999, which is incorporated by reference herein its entirety.

Consequently, the methods of the present invention also encompass administering therapeutically-effective amounts of a deuterated form of Compound 1, or a pharmaceutically-acceptable salt thereof, and pharmaceutical compositions, pharmaceutical dosage forms, and medicaments comprising a deuterated form of Compound 1.

### EXAMPLES

The following examples are illustrative, but not limiting, of the methods and formulations of the present invention. Other suitable modifications and adaptations of the variety of conditions and parameters normally encountered in chemotherapeutic methods and in drug formulation, and which would be within the purview of those skilled in the art, are within the spirit and scope of the invention.

### Example 1

### Efficacy of Compound 1 in Murine Xenograft Models

Mice were implanted with specified numbers of cells from one of a variety of cancer cell types, including MV-4-11, HT29, DU-145, NCI-H69, OVCAR-3, BT-474, NCI-N87, OPM-2, B16, A549, Colo205, K-562, NCI-H460, and MIA PaCa-2, to create xenografts in athymic nude mice. The resulting xenografted tumor were allowed to grow to a specified size before the xenografted mice were dosed orally with either vehicle alone (30% to 40% Captisol® (CyDex Pharmaceuticals, Inc.; Lenexa, Kans.) in phosphate buffer), or Compound 1 in vehicle in a variety of doses and regimens as outlined in Table 1.

Among other results, mice dosed with 200 mg/kg of Compound 1 displayed activity that ranged from 50% to 100% tumor growth inhibition (TGI), and up to 50% tumor regression (Table 1). Animals showed no significant weight loss. As shown in Table 1, Compound 1 is efficacious in multiple murine xenograft models.

TABLE 1
Efficacy of Compound 1 in a Variety of Murine Xenograft Models

| Xenograft | Compound 1 | | | |
|---|---|---|---|---|
| Model | Oral Dose | | Regimen | Response |
| NCI-N87 | 200 | mg/kg | 5-days-on/2-days- | 44% regression after 3 cycles |
| Her2+ gastric | | | off | |
| cancer | 50, 75, 125, 200 | mg/kg | Daily for 21 days | 50% regression (200 mg/kg); |
| | | | | 100% TGI (125 mg/kg) |
| | 200, 400 | mg/kg | Daily for 21 days | 40% regression, 7% |
| | | | (200), QOD (200), | regression, 100% TGI, 95% |
| | | | twice weekly (200, | TGI |
| | | | 400) | |
| | 200 | mg/kg | Weekly (Days 1, 8, | 54% TGI (p = 0.1) Day 21 |
| | | | 15) | |
| | 100 | mg/kg | Daily for 21 days | 82% TGI (p = 0.003) Day 21 |
| | 100 | mg/kg | Twice daily for 21 | 50% regression (p < 0.0001) |
| | | | days | Day 21 |
| HT-29 colon | 200 | mg/kg | 5-days-on/2-days- | 68% TGI after 3 cycles |
| cancer | | | off | |
| DU-145 | 200 | mg/kg | 5-days-on/2-days- | Average of ~80% TGI after 5 |
| prostate cancer | | | off | cycles |
| NCI-H69 small | 200 | mg/kg | Daily for 27 days | Extended time to 1,500 mm³ |
| cell lung cancer | | | | (p = 0.01); 8/10 treated mice |
| | | | | have tumors <1,500 mm³ |
| | | | | compared to 2/9 for vehicle |
| | | | | arm |
| MV-4-11 acute | 200 | mg/kg | Daily for 21 days | 50% regression |
| myeloid | | | | |
| leukemia | | | | |
| OVCAR-3 | 150, 200 | mg/kg | Daily for 35 days | 84%, 97% TGI |
| ovarian cancer | | | | |
| B16 melanoma | 200 | mg/kg | Daily for 8 days | 50% TGI |
| allograft | | | | |
| BT-474 HER2+ | 200 | mg/kg | Daily for 70 days | 17% regression |
| breast cancer | | | | |
| MIA PaCa-2 | 150, 200 | mg/kg | Daily for 21 days | 67%, 95% TGI |
| pancreatic | | | | |
| cancer | | | | |
| A549 non-small | 150, 200 | mg/kg | Daily for 21 days | 88% TGI (p = 0.26) 150 mg/kg; |
| cell lung cancer | | | | 16% regression |
| | | | | (p = 0.06) 200 mg/kg |
| Colo205 colon | 150, 200 | mg/kg | Daily for 21 days | 72% TGI (p = 0.3) 150 mg/kg; |
| cancer | | | | 87% TGI (p = 0.03) 200 mg/kg |
| OPM-2 | 200 | mg/kg | Daily for 21 days | 71% TGI (p = 0.34) |
| multiple | | | | |
| myeloma | | | | |
| K-562 | 150, 200 | mg/kg | Daily for 18 days | 82% TGI (p = 0.14) 150 mg/kg, |
| erythroleukemia | | | | 97% TGI (p = 0.05) |
| | | | | 200 mg/kg |
| NCI-H460 non- | 200 | mg/kg | Daily for 21 days | 61% TGI (p = 0.05); extended |
| small cell lung | | | | time to 1,500 mm³ (p = 0.006), |
| cancer | | | | 4/8 treated mice have tumors |
| | | | | <1,500 mm³ compared to 1/10 |
| | | | | for vehicle arm |

### Example 2

### Efficacy and Safety Comparison of Compound 1 to SNX-5422 in a Murine Xenograft Model

The efficacy of Compound 1 was compared to that of SNX-5422 (Serenex, Pfizer, Inc.) on the growth of NCI-N87 (Her2+) human gastric cancer cells as a xenograft in athymic nude mice model. Animals were dosed with either 200 mg/kg of Compound 1 on days 1 through 21 or 40 mg/kg of SNX-5422 thrice weekly for three weeks. Tumor volumes and body weights were determined from Days 1 to 39.

Five million NCI-N87 cells were implanted subcutaneously in the right flank of female nude mice (Hsd:athymic nude-Foxn1^{nu}). When the median tumor volume was approximately 123 mm³, mice were randomized into three cohorts of ten animals. One cohort was dosed orally with vehicle, one cohort was dosed orally with Compound 1 (200 mg/kg) formulated in vehicle, and one cohort was dosed orally with SNX-5422 (40 mg/kg) formulated in dimethylacetamide/polyethylene glycol 300. Vehicle and Compound 1 were administered on days 1-21 and SNX-5422 was administered thrice weekly on Days 1, 3, 5, 8, 10, 12, 15, 17 and 19. The mice were observed daily for mortality and signs of toxicity.

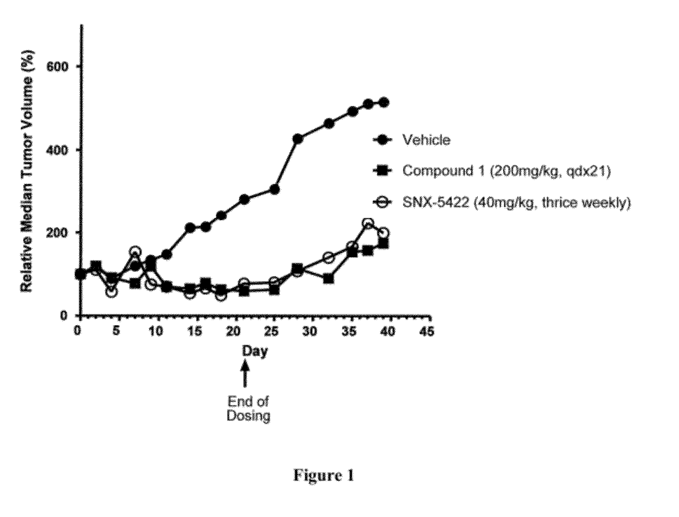

In the N-87 xenograft model (FIG. 1), the median tumor volume of animals dosed with Compound 1 at 200 mg/kg (maximum tolerated dose (“MTD”), 1 death on day 21) decreased by 40% by day 21. Similarly, the median tumor volume of animals treated with SNX-5422 (MTD, 2 deaths; day 7, day 20) decreased by 22% on day 21. The maximal reduction in median body weight of the cohorts dosed with Compound 1 was 13% of the pretreatment weight on day 25. The maximal reduction in median body weight of the animals dosed with SNX-5422 was 17% on day 18. Compound 1 significantly inhibited growth of a human (Her2+) gastric carcinoma xenograft in athymic nude mice when dosed orally at 200 mg/kg on a once-a-day schedule. Compound 1 was as effective as SNX-5422 in inhibiting tumor growth. Compound 1 administration caused no significant reduction in body weight.

### Example 3

### Phase I Clinical Trial of Orally Administered Compound 1

A human clinical study was initiated at a starting dose of 50 mg/m². Subjects had recurrent cancer refractory to available systemic therapy. Compound 1 was administered daily by mouth in tablet form for 21 consecutive days in a 28 day cycle to each enrolled subject. For determination of drug levels in humans, plasma was collected prior to drug administration and 0.5, 1, 2, 3, 4, 6, 8, and 24 hours post-dosing on cycle 1 day 1, and cycle 1 day 21. Plasma was collected pre-dose only for cycle 1 day 8. Peripheral blood mononuclear cells (PBMCs) were collected prior to drug administration, and typically 8 and 24 hours post-dose on cycle 1 day 1 and cycle 1 day 21 in order to quantify Hsp70 protein levels as an exploratory biomarker. Patients were men and women with metastatic cancer, ranged in age from 45 to 85 years, and received doses ranging from 50 mg/m² to 340 mg/m², with a total daily dose of 100-740 mg (Tables 2A and 2B). In Tables 2A and 2B, for the BID (i.e., twice daily dosing) regimen, the AUC_{(0-inf)} is based on a single 12-hour dosing interval.

TABLE 2A

| | | Subject | Dose | Dose | t_{1/2} | Tₘₐₓ | Cₘₐₓ |
|---|---|---|---|---|---|---|---|
| Day | Cohort | No. | (mg/m²) | (mg) | (hr) | (hr) | (ng/mL) |
| Day 01 | 1 | 101 | 50 | 100 | 8.5 | 2.0 | 1904 |
| Day 01 | 2 | 201 | 100 | 160 | 9.8 | 2.0 | 2949 |
| Day 01 | 3 | 301 | 165 | 300 | 11.5 | 4.0 | 6869 |
| Day 01 | 4 | N = 6 | 245 | 340-540 | 10.8 | 2.5 | 7837 |
| Day 01 | 5 | N = 2 | 340 | 480-620 | 14.1 | 3.0 | 16274 |
| Day 01 | 5 (BID) | N = 6 | 340 | 270-370 | 7.5 | 2.0 | 8122 |
| Day 01 | 6 (BID) | N = 4 | NA | 240 | 5.5 | 1.0 | 6253 |
| Day 19 | 1 | 101 | 50 | 100 | 13.4 | 1.0 | 1701 |
| Day 21 | 2 | 201 | 100 | 160 | 11.2 | 6.0 | 3182 |
| Day 21 | 3 | 301 | 165 | 300 | 14.0 | 8.0 | 11789 |
| Day 21 | 4 | N = 6 | 176-245 | 340-540 | 13.2 | 2.0 | 9266 |
| Day 21 | 5 | 503 | 340 | 620 | 19.3 | 1.0 | 25587 |
| Day 21 | 5 (BID) | 508 | 340 | 330 | 6.7 | 1.0 | 10967 |
| Day 21 | 6 (BID) | N = 2 | NA | 240 | 6.2 | 1.5 | 9199 |

TABLE 2B

| | | AUC₍₀₋₁₂₎ | AUC₍₀₋₂₄₎ | AUC_{(0-inf)} | | |
|---|---|---|---|---|---|---|
| | | (hr*ng/ | (hr*ng/ | (hr*ng/ | CL/F | V_{z}/F |
| Day | Cohort | mL) | mL) | mL) | (mL/hr) | (mL) |
| Day 01 | 1 | | 20636 | 24405 | 4098 | 50538 |
| Day 01 | 2 | | 29869 | 36843 | 4343 | 61645 |
| Day 01 | 3 | | 91824 | 124744 | 2405 | 39963 |
| Day 01 | 4 | | 99124 | 142833 | 3118 | 61438 |
| Day 01 | 5 | | 232951 | 334038 | 2500 | 51394 |
| Day 01 | 5 (BID) | 62145 | | 107255 | 2517 | 28398 |
| Day 01 | 6 (BID) | 38228 | | 55014 | 5956 | 39579 |
| Day 19 | 1 | | 21992 | 30826 | 4547 | 87981 |
| Day 21 | 2 | | 49805 | 67325 | 3213 | 52005 |
| Day 21 | 3 | | 199749 | 308201 | 1502 | 30432 |
| Day 21 | 4 | | 126959 | 201766 | 3509 | 77941 |
| Day 21 | 5 | | 362773 | 609969 | 1323 | 36756 |
| Day 21 | 5 (BID) | 69533 | | 96340 | 4746 | 45745 |
| Day 21 | 6 (BID) | 67619 | | 95102 | 4003 | 34650 |

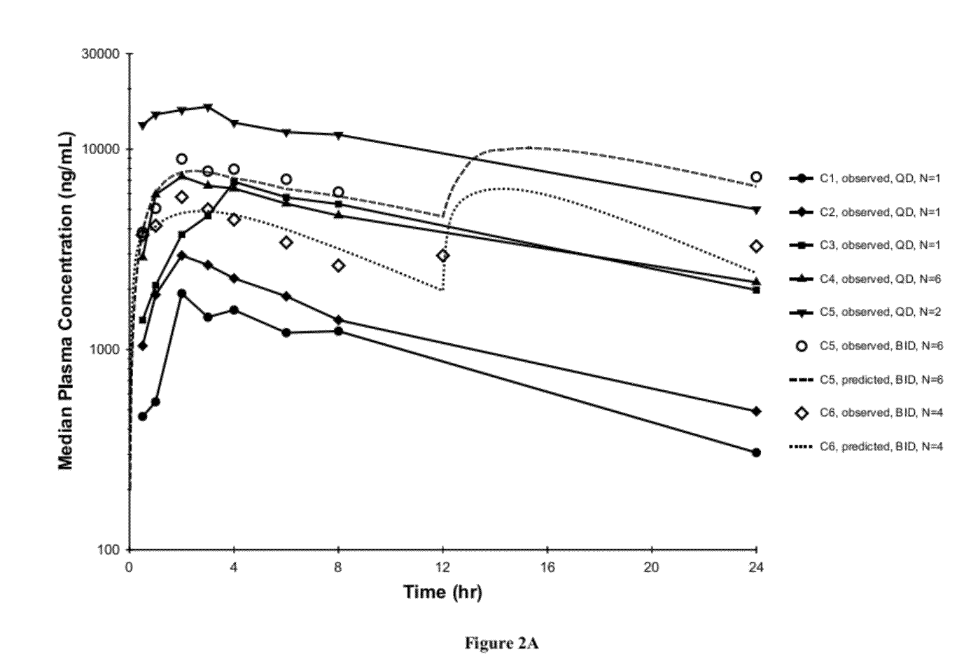

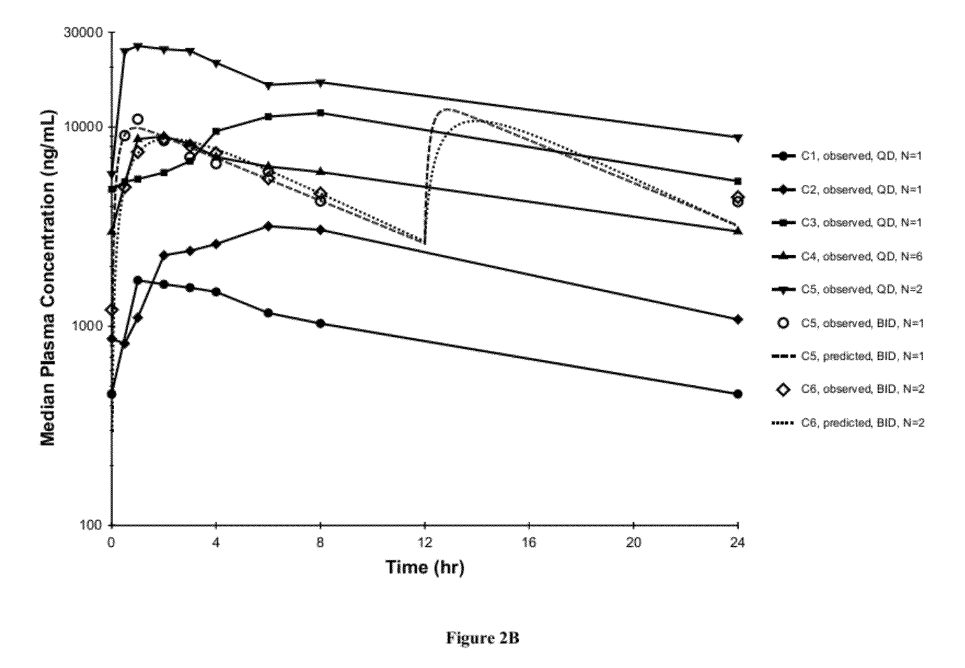

There was a more-than-proportional increase in drug levels with increasing dose (FIGS. 2A and 2B). Plasma Cₘₐₓ and AUC₍₀₋₂₄₎ in the patient dosed at 165 mg/m² (11789 ng/mL and 199,749 hr*ng/mL at day 21) are comparable in magnitude to those achieved in tumor-bearing mice after a single dose of 200 mg/kg (21841 ng/mL and 135,779 hr*ng/mL, respectively). The drug level achieved in humans has been found to have anti-tumor activity in multiple murine xenograft models (see Tables 1 and 3) when achieved in mice. Patients have completed between 1 to 13 (28-day) cycles. No dose limiting toxicities have been reported to date.

TABLE 3

| | | | t_{1/2} | Tₘₐₓ | Cₘₐₓ | AUC₍₀₋₂₄₎ |
|---|---|---|---|---|---|---|
| Species | Dose | Day | (hr) | (hr) | (ng/mL) | (hr*ng/mL) |
| N-87 | 200 mg/kg | Single | 4.8* | 4.0* | 21841* | 135779* |
| tumor- | | Dose | | | | |
| bearing | | | | | | |
| nude mice | | | | | | |

*median values (n = 6)

These studies show that Compound 1 is orally bioavailable in human cancer patients. Further, the pharmacokinetic properties and drug concentrations achieved in human patients are similar to those observed in efficacious mouse xenograft experiments.

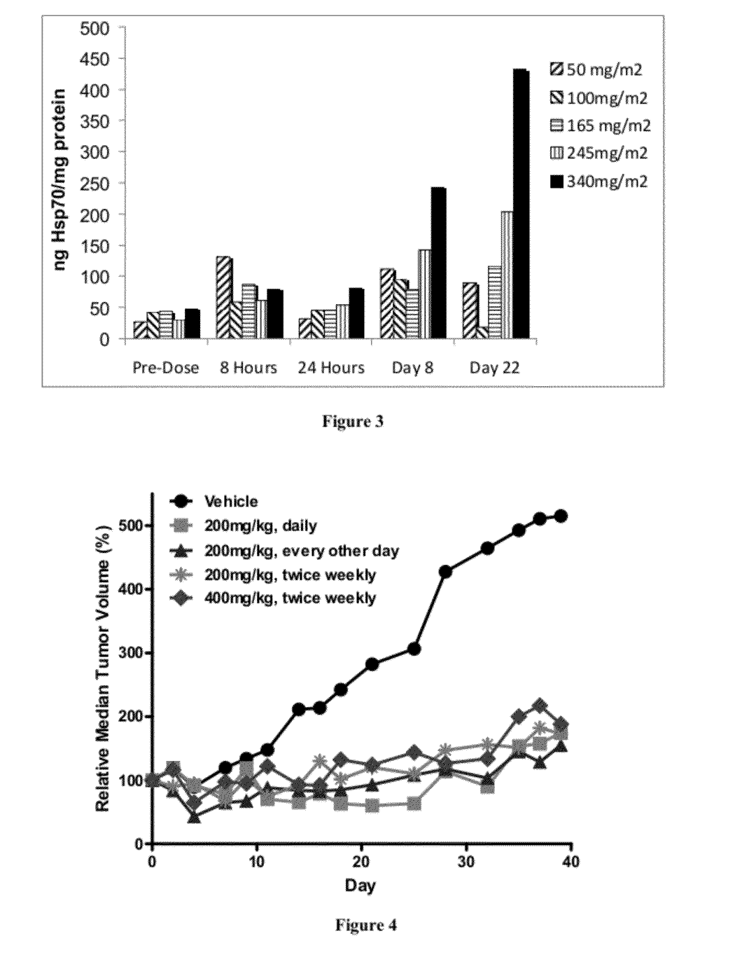

Importantly, Hsp90 inhibition disrupts the sequestration of the heat shock transcription factor Hsf1 by Hsp90 resulting in the expression of Hsp70. Therefore, induction of Hsp70 expression in PBMCs is a potentially useful clinical biomarker with which to monitor Hsp90 inhibition. Hsp70 levels in protein extracts derived from PBMCs showed an increase 8 hours after the first dose, which was sustained on day 8 and day 22 (see FIG. 3). Hsp70 protein levels were determined by an ELISA with a Hsp 70 ELISA kit (catalog #EKS-700B, Assay Designs, Stressgen, Ann Arbor, Mich.) as per the manufacturer's protocol.

### Example 4

### Efficacy of Alternate Dosing Schedules

Xenografted mice bearing N-87 tumors (median volume ˜125 mm³) were dosed orally with Compound 1 for three weeks with the dosing schedules indicated in FIG. 4.

Compound 1 was found to be effective when dosed daily (40% regression), once every-other-day (7% regression), twice-weekly (89% TGI), or 400 mg/kg twice-weekly (87% TGI) on Day 21 (p<0.0001). Tumor growth inhibition (TGI) was monitored up to Day 39. Although the 200 mg/kg daily schedule was significantly more effective (p<0.03) than all other schedules at the end of dosing on Day 21, there was no significant difference in TGI (79 to 87%) between the various schedules at the end of the study on Day 39.

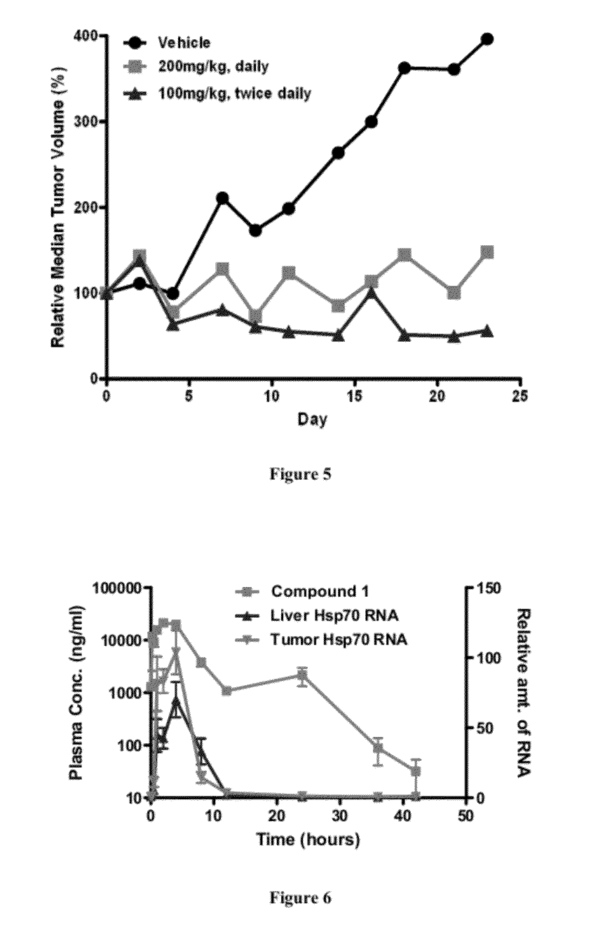

Additionally, xenografted mice bearing N-87 tumors (median volume ˜115 mm³) were dosed orally with Compound 1 for twenty-one days at 200 mg/kg, once daily or 100 mg/kg, twice-daily as indicated in FIG. 5. Tumor growth inhibition (TGI) was monitored up to Day 39. Compound 1 was effective when dosed twice-daily at 100 mg/kg (50% regression, p<0.0001).

### Example 5

### Hsp70 Induction by Compound 1

Xenografted mice bearing N-87 tumors (median volume ˜425 mm³) were given a single oral dose of Compound 1 (200 mg/kg). Blood, tumor and liver samples (n=6) were collected at 0.25, 0.5, 1, 2, 4, 8, 12, 24, 36 and 48 h post-dose and used to determine Compound 1 concentrations in the plasma and Hsp70 RNA levels in tumor and liver samples (See FIG. 6). Hsp70 RNA levels were determined by qRT-PCR. Hsp70 RNA is induced by ˜70- and ˜110-fold in liver and tumor tissue, respectively, 4 hours post-dose. This induction was found to revert to baseline by 12 hours post-dose.

### Example 6

### Efficacy of Compound 1 in a Murine Xenograft Model Compared to Erlotinib

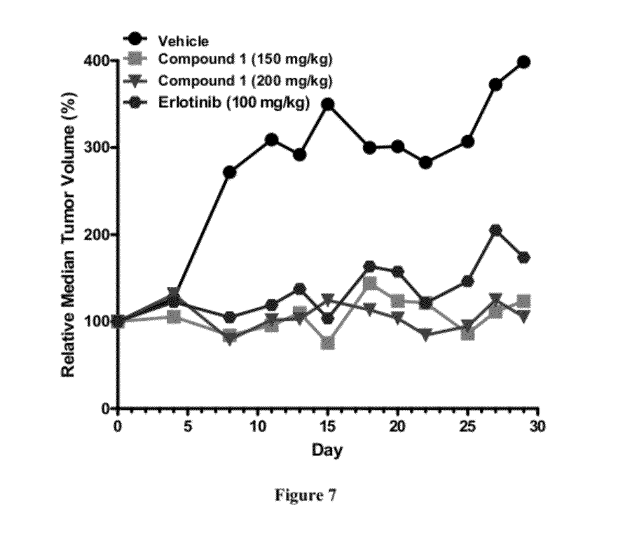

Xenografted mice bearing A549 non-small cell lung cancer tumors (median volume 100 mm³) were dosed orally, once daily, with Compound 1 or erlotinib (EGFR1) for 21 days with the doses indicated in FIG. 7.

Compound 1 was effective when dosed at 200 mg/kg (16% regression) or 150 mg/kg (88% TGI) compared to erlotinib at its maximum tolerated dose (88% TGI) on Day 22.

### Example 7

### Efficacy of Compound 1 in a Murine Xenograft Model Compared to 5-Fluorouracil

Xenografted mice bearing MIA PaCa-2 pancreatic tumors (median volume ˜150 mm³) were dosed orally with Compound 1 (150 mg/kg or 200 mg/kg) daily for 15 days or weekly with 5-Fluorouracil (100 mg/kg, ip).

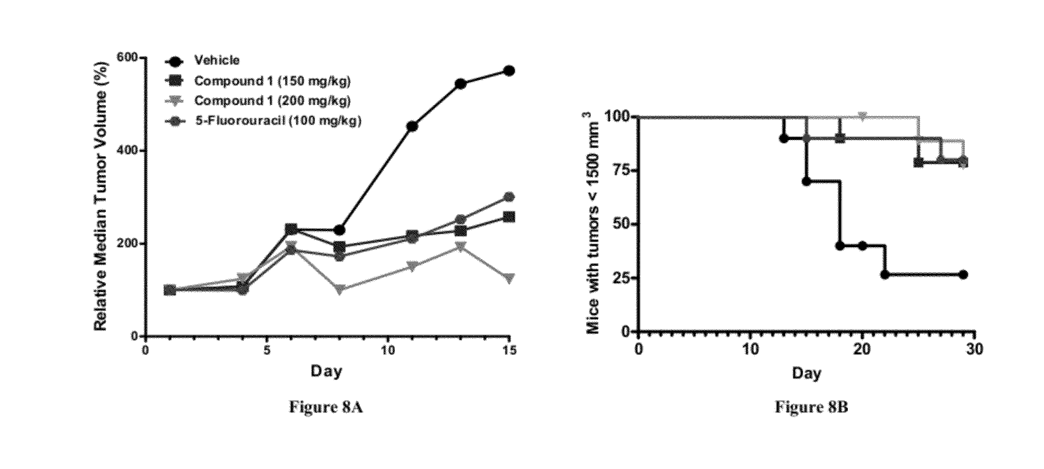

Compound 1 was effective when dosed at 200 mg/kg or 150 mg/kg (95% TGI and 67% TGI, see FIGS. 8A and 8B) compared to 5-Fluorouracil (58% TGI) on Day 15. The median time to tumor volume >1,500 mm³ was 18 days for the vehicle group and >29 days for all treatment groups (p<0.05). Tumor growth inhibition observed with Compound 1 in murine MIA PaCa-2 xenografted mice compared favorably to that observed with 5-fluorouracil (58% TGI).

### Example 8

### Oral Pharmacokinetics of Compound 1 in a Single Dose Versus Two Doses

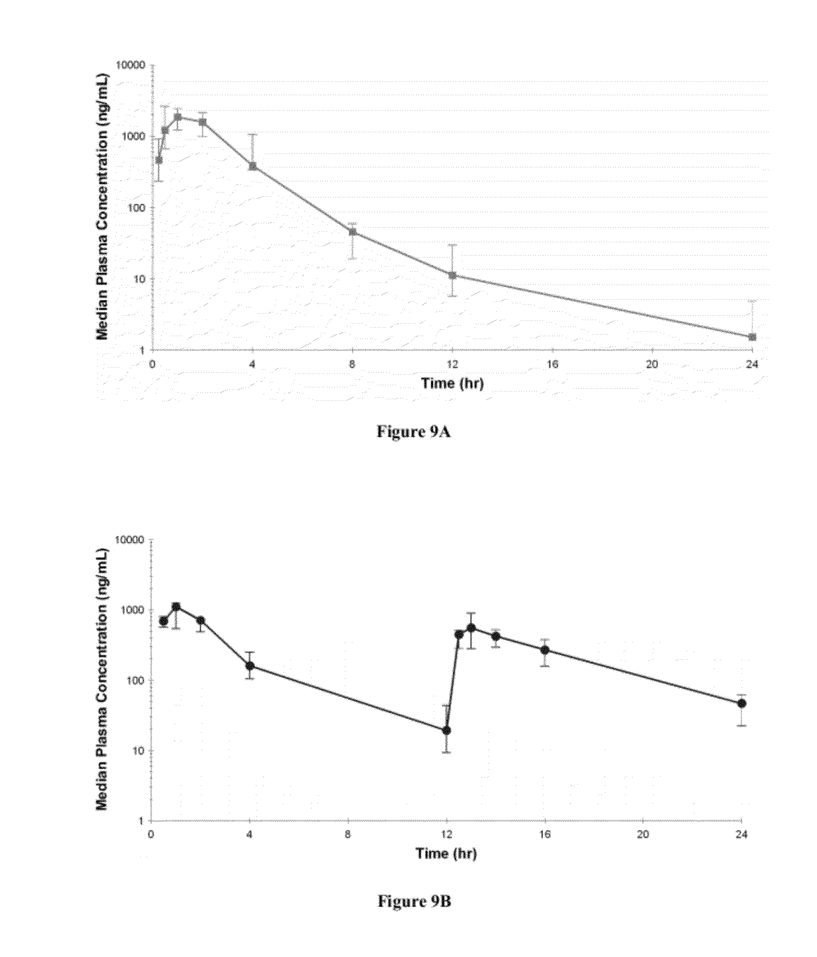

Female Sprague Dawley rats (n=4 or 5) were dosed orally once with 50 mg/kg (See FIG. 9A) or twice with 25 mg/kg (See FIG. 9B) of Compound 1. Pharmacokinetic parameters are outlined in Table 4 below. Two doses of 25 mg/kg, twelve hours apart, give similar exposure as a single dose of 50 mg/kg. Plasma concentration of Compound 1 at 24 h is significantly higher with BID dosing. Effective plasma concentrations of Compound 1 were maintained with twice a day dosing.

TABLE 4

| Dose | | Cₘₐₓ | AUC₍₀₋₂₄₎ | C₂₄ₕᵣ |
|---|---|---|---|---|
| (mg/kg) | Regimen | (ng/mL) | (hr * ng/mL) | (ng/mL) |
| 50 | Single | 2122 | 5835 | 1.5 |
| | dose | | | |
| 25 | Two doses | 1113 | 5123 | 46.7 |
| | 12 h apart | | | |

### Example 9

### Solid Pharmaceutical Dosage Form Comprising 20 mg of Compound 1

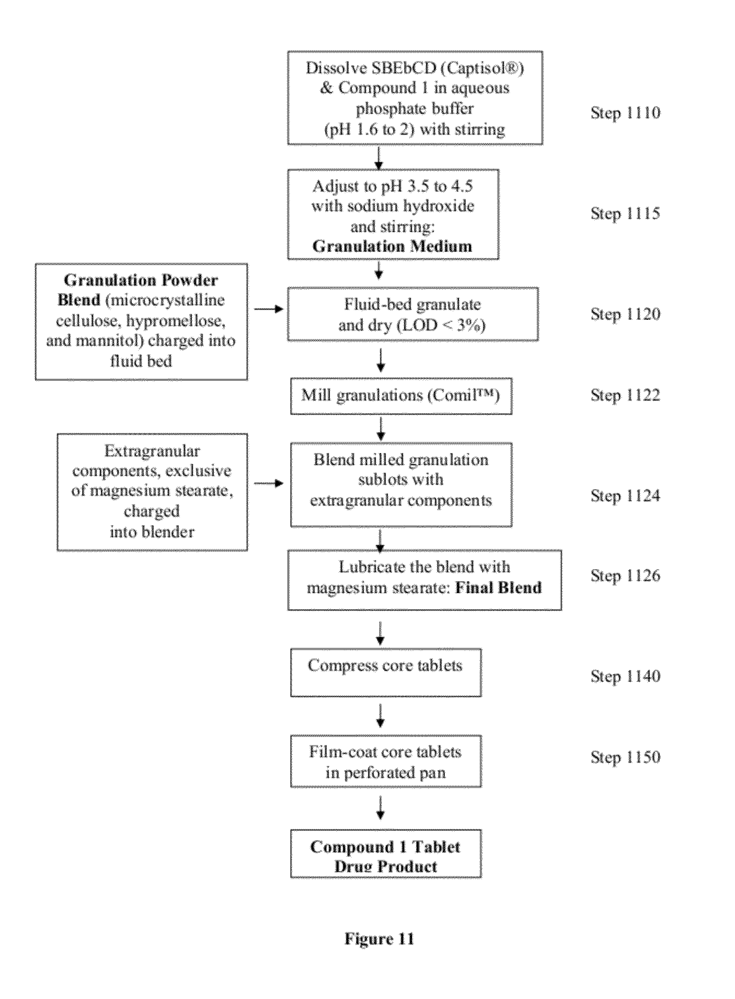

A solid pharmaceutical dosage form comprising 20 mg of Compound 1 was prepared using the components of Table 5 in the quantities listed. Table 5 describes an exemplary tablet formulation comprising 20 mg of Compound 1 prepared according to the process illustrated in FIG. 11 and discussed in more detail below.

TABLE 5

| | | Batch Formula: |
|---|---|---|
| | Component | Theoretical Amounts |
| | Weight | for 6,688 Tablets |
| Component | (mg/tablet) | (g/component) |
| Intragranular^{a} | | |
| Granulation Powder Blend: | | |
| Microcrystalline Cellulose (Avicel | 113.50 | 759.1 |
| PH302) | | |
| Mannitol | 113.50 | 759.1 |
| (Pearlitol ® 160C (Roquette)) | | |
| Hypromellose (Methocel ™ E5P | 13.00 | 86.9 |
| LV) | | |
| Granulation Medium: | | |
| Compound 1^{b} | 20.00 | 133.8 |
| SBEbCD (Captisol ®) | 250.00 | 1672.0 |
| Sodium Phosphate Monobasic, | 2.43 | 16.3 |
| Anhyd. | | |
| NaOH, pellets | 5.24 | 35.0 |
| Phosphoric Acid^{c} | 13.50^{d} | 102.1^{e} |
| Purified Water, USP | n/a^{f} | 2498.5 |
| Intragranular Total: | 531.2 | 3552.3 |
| Extragranular | | |
| Microcrystalline Cellulose | 55.70 | 372.5 |
| (Avicel PH302) | | |
| Croscarmellose Sodium Type A | 18.40 | 123.1 |
| (Ac-Di-Sol) | | |
| Colloidal Silicon Dioxide (Cab- | 3.10 | 20.7 |
| O-Sil M-5P) | | |
| Magnesium Stearate | 4.60 | 30.8 |
| (Vegetable; non-Bovine) | | |
| Extragranular Total: | 81.8 | 547.1 |
| Film-Coat | | |
| Opadry II Pink, 85F94592 | 24.5 | 164 |
| Water | n/a^{f} | 929 |
| Total, core tablet: | 613.0 | 4099.4 |
| Total, film-coated tablet: | 637.5 | 4263.4 |

^{a}divided into three equal sublots for fluid-bed granulation processing
^{b}actual amount adjusted per purity of Compound 1 drug substance
^{c}phosphoric acid is 88.4% solids, by weight
^{d}solids only; water not included
^{e}water content is included
^{f}removed during processing

In the exemplary 20 mg tablets, a granulation medium was prepared by dissolving, with mixing, SBEbCD (Captisol®; CyDex Pharmaceuticals, Inc.; Lenexa, Kans.) into an aqueous, acidic (pH approximately 2) phosphate buffer prepared from water, sodium phosphate monobasic, and phosphoric acid. See Step 1110 of FIG. 11. This solution was gently heated to approximately 40° C., and Compound 1 (amount added was adjusted based on the purity of the dried Compound 1 drug substance) was slowly added with continuous mixing until complete dissolution was achieved. The granulation medium was then cooled to room temperature. The pH of the granulation medium was adjusted to pH 3.5 to 4.5 with a 2N sodium hydroxide solution as determined with a calibrated pH meter. See Step 1115 of FIG. 11.

The components of the intragranular granulation powder blend were subdivided into three equal portions (sub-batch A, B, C) and sieved (20 mesh). Each sub-batch was granulated by a fluid-bed process using approximately ⅓ of the granulation medium (discussed above) applied from a top-spray configuration. Once all granulation medium was applied, the granulation mixture was dried in the fluid-bed until the product weight loss on drying at 105° C. in a moisture analyzer was <3% (i.e., LOD <3%). See Step 1120 of FIG. 11.

The granulation sub-batches were milled and combined in a bin blender. See Step 1122 of FIG. 11. The dried milled blended granulation was analyzed (High Performance Liquid Chromatography) for potency, particle size distribution, and density (bulk and tapped).

The extragranular components, exclusive of magnesium stearate, were sieved (20 mesh) and blended in a bin blender with the combined granulation from above. See Step 1124 of FIG. 11. The amounts of the extragranular components were adjusted based on the actual yield and potency of the combined granulation from above.

The magnesium stearate was sieved (30 mesh) and charged into the bin blender with the blended components described just previously. See Step 1126 of FIG. 11. After blending, the final blend was analyzed for density (bulk and tapped).

The final blend was compressed into core tablets on a multistation press fitted with modified oval tooling (0.3375″×0.675″). See Step 1140 of FIG. 11. The weight (target core tablet weight was 613 mg/tablet) and hardness (13 to 19 Kp) were monitored throughout the compression procedure. Friability was determined on core tablets meeting the weight and hardness specifications.

The core tablets were pan coated with an Opadry® II Pink (polyvinyl alcohol, titanium dioxide, polyethylene glycol, talc, iron oxide red) suspension to a 4% target weight gain to complete the manufacture of the exemplary 20 mg Compound 1 drug product. See Step 1150 of FIG. 11.

Table 6 lists the general function(s) of each component of the exemplary 20 mg tablet.

TABLE 6

| Component | Function |
|---|---|
| Compound 1 | Drug Substance |
| SBEbCD (Captisol ®) | Solubilizing Agent |
| Opadry II Pink, 85F94592 | Film Coat |
| Colloidal Silicon Dioxide | Glidant |
| (Cab-O-Sil M-5P) | |
| Croscarmellose Sodium | Disintegrant |
| (Ac-Di-Sol) | |
| Mannitol (Pearlitol ® 160C) | Diluent |
| Microcrystalline Cellulose | Diluent/Disintegrant |
| (Avicel PH302) | |
| Methocel ™ E5P LV | Binder |
| (hypromellose 2910; hydroxypropyl | |
| methylcellulose) | |
| Magnesium Stearate - Non-Bovine | Lubricant |
| Sodium Phosphate Monobasic | Buffering agent |
| Phosphoric Acid | Acidifying agent |
| NaOH | Neutralizing agent |

### Example 10

### Solid Pharmaceutical Dosage Form Comprising 40 mg of Compound 1

A solid pharmaceutical dosage form comprising 40 mg Compound 1 was prepared using the components of Table 7 in the quantities listed. Table 7 lists an exemplary 40 mg Compound 1 tablet formulation prepared according to the process illustrated in FIG. 11 and discussed in more detail below.

TABLE 7

| | | Batch Formula: |
|---|---|---|
| | Component | Theoretical Amounts |
| | Weight | for 6,688 Tablets |
| Component | (mg/tablet) | (g/component) |
| Intragranular^{a} | | |
| Granulation Powder Blend: | | |
| Microcrystalline Cellulose (Avicel | 227.00 | 1518.2 |
| PH302) | | |
| Mannitol | 227.00 | 1518.2 |
| (Pearlitol ® 160C) | | |
| Hypromellose (Methocel ™ E5P | 26.00 | 173.9 |
| LV) | | |
| Granulation Medium: | | |
| Compound 1^{b} | 40.00 | 267.5 |
| SBEbCD (Captisol ®) | 500.00 | 3344.0 |
| Sodium Phosphate Monobasic, | 4.86 | 32.5 |
| Anhyd. | | |
| NaOH, pellets | 10.48 | 70.1 |
| Phosphoric Acid^{c} | 27.00^{d} | 204.3^{e} |
| Purified Water, USP | n/a^{f} | 4999.7 |
| Intragranular Total: | 1062.34 | 7105.1 |
| Extragranular | | |
| Microcrystalline Cellulose | 111.40 | 745.0 |
| (Avicel PH302) | | |
| Croscarmellose Sodium Type A | 36.80 | 246.1 |
| (Ac-Di-Sol) | | |
| Colloidal Silicon Dioxide (Cab- | 6.20 | 41.5 |
| O-Sil M-5P) | | |
| Magnesium Stearate | 9.20 | 61.5 |
| (Vegetable; non-Bovine) | | |
| Extragranular Total: | 163.60 | 1094.1 |
| Film-Coat | | |
| Opadry II Pink, 85F94592 | 49.0 | 328.0 |
| Water | n/a^{f} | 1858 |
| Total, core tablet: | 1226 | 8199.2 |
| Total, film-coated tablet: | 1275 | 8527.2 |

^{a}divided into six equal sublots for fluid-bed granulation processing
^{b}actual amount adjusted per purity of Compound 1 drug substance
^{c}phosphoric acid is 88.4% solids, by weight
^{d}solids only; water not included
^{e}water content is included
^{f}removed during processing

In the exemplary 40 mg tablets, three sub-batches of a granulation medium were prepared by dissolving, with mixing, SBEbCD (Captisol®; CyDex Pharmaceuticals, Inc.; Lenexa, Kans.) into an aqueous, acidic (pH approximately 2) phosphate buffer prepared from water, sodium phosphate monobasic, and phosphoric acid. See Step 1110 of FIG. 11. This solution was gently heated to approximately 40° C., and Compound 1 (amount added was adjusted based on the purity of the dried Compound 1 drug substance) was slowly added with continuous mixing until complete dissolution was achieved. The granulation medium was then cooled to room temperature. The pH of the granulation medium was adjusted to pH 3.5 to 4.5 with a 2N sodium hydroxide solution as determined with a calibrated pH meter. See Step 1115 of FIG. 11.

The components of the intragranular granulation powder blend were subdivided into six equal portions (sub-batch A, B, C, D, E, and F) and sieved (20 mesh). Each sub-batch was granulated by a fluid-bed process using approximately ⅙th of the granulation medium (discussed above as being prepared in three sub-batches) applied from a top-spray configuration. Once all granulation medium was applied, the granulation mixture was dried in the fluid-bed until the product weight loss on drying at 105° C. in a moisture analyzer was <3% (i.e., LOD <3%). See Step 1120 of FIG. 11.

The granulation sub-batches were milled and combined in a bin blender. See Step 1122 of FIG. 11. The dried milled blended granulation was analyzed (High Performance Liquid Chromatography) for potency, particle size distribution, and density (bulk and tapped).

The extragranular components, exclusive of magnesium stearate, were sieved (20 mesh) and blended in a bin blender with the combined granulation from above. See Step 1124 of FIG. 11. The amounts of the extragranular components were adjusted based on the actual yield and potency of the combined granulation from above.

The magnesium stearate was sieved (30 mesh) and charged into the bin blender with the blended components described just previously. See Step 1126 of FIG. 11. After blending, the final blend was analyzed for density (bulk and tapped).

The final blend was compressed into core tablets on a multistation press fitted with modified oval tooling (0.3375″×0.8200″). The weight (target core tablet weight was 1,226 mg/tablet) and hardness (15 to 21 Kp) were monitored throughout the compression procedure. See Step 1140 of FIG. 11. Friability was determined on core tablets meeting the weight and hardness specifications.

The core tablets were pan coated with an Opadry® II Pink (polyvinyl alcohol, titanium dioxide, polyethylene glycol, talc, iron oxide red) suspension to a 4% target weight gain to complete the manufacture of the exemplary 40 mg Compound 1 drug product. See Step 1150 of FIG. 11.

The general function(s) of each component of the exemplary 40 mg tablet were the same as those listed for the exemplary 20 mg tablet of Example 9 in Table 6.

### Example 11

### Solid Pharmaceutical Dosage Form Comprising 75 mg of Compound 1

A solid pharmaceutical dosage form comprising 75 mg of Compound 1 can be prepared using the components of Table 7 with the quantities of Compound 1 and the SBEbCD increased by a factor of 1.875, and following a process similar to that discussed in Example 10. An exemplary tablet comprising 75 mg of Compound 1 is produced.

### Example 12

### Liquid Pharmaceutical Dosage Form Comprising Compound 1

A liquid pharmaceutical dosage form comprising Compound 1 can be prepared using the granulation medium components of either Table 5 or Table 7. An abbreviated process initially similar to that discussed in Example 9 or Example 10 is followed. An exemplary process is further described below.

Granulation medium is prepared by dissolving, with mixing, SBEbCD (Captisol®; CyDex Pharmaceuticals, Inc.; Lenexa, Kans.) into an aqueous, acidic (pH approximately 2) phosphate buffer prepared from water, sodium phosphate monobasic, and phosphoric acid. This solution is gently heated to approximately 40° C., and Compound 1 (amount added is adjusted based on the purity of the dried Compound 1 drug substance) is slowly added with continuous mixing until complete dissolution is achieved. The granulation medium is then cooled to room temperature. The pH of the granulation medium is adjusted to pH 3.5 to 4.5 with a 2N sodium hydroxide solution as determined with a calibrated pH meter.

The granulation medium is sterile filtered with a 0.2 μm disposable filter unit and stored. The dosage of individual units may be adjusted by the volume of individual storage units.

Alternatively, after sterile filtration, the granulation medium is freeze-dried or lyophilized to form a reconstitutable powder/cake from which a liquid dosage form can be prepared at the point of use by or for a patient through the addition of water for injection.

The solution of Compound 1 dissolved in aqueous SBEbCD (Captisol®; CyDex Pharmaceuticals, Inc.; Lenexa, Kans.) described in step 1010 of FIG. 10 or Step 1115 of FIG. 11 may be dosed orally without prior sterile filtration. The solution may be dosed parenterally with sterile filtration. The freeze-dried or lyophilized product is suitable for either oral or parenteral administration.

### Example 13

### Granular Pharmaceutical Dosage Forms Comprising Compound 1

A granular pharmaceutical dosage form comprising Compound 1 can be prepared using the intragranular components of either Table 5 or Table 7. In addition, a granular pharmaceutical dosage form comprising Compound 1 can be prepared using some, not all, of the intragranular powder blend components of either Table 5 or Table 7. An abbreviated process initially similar to that discussed in Example 9 or Example 10 is followed.

Granulation medium is prepared by dissolving, with mixing, SBEbCD (Captisol®; CyDex Pharmaceuticals, Inc.; Lenexa, Kans.) into an aqueous, acidic (pH approximately 2) phosphate buffer prepared from water, sodium phosphate monobasic, and phosphoric acid. This solution is gently heated to approximately 40° C., and Compound 1 (amount added is adjusted based on the purity of the dried Compound 1 drug substance) is slowly added with continuous mixing until complete dissolution is achieved. The granulation medium is then cooled to room temperature. The pH of the granulation medium is adjusted to pH 3.5 to 4.5 with a 2N sodium hydroxide solution as determined with a calibrated pH meter.

The components of the intragranular granulation powder blend are subdivided as necessary and sieved (20 mesh). The granulation powder blend is granulated by a fluid-bed process using the granulation medium applied from a top-spray configuration. Once all granulation medium is applied, the granulation mixture is dried in the fluid-bed until the product weight loss on drying at 105° C. in a moisture analyzer was <3% (i.e., LOD <3%).

Optional alternative step A: Capsules, such as hard gelatin capsules can be filled with the granulation mixture. The dosage of individual capsules can be determined by the size of the capsule filled and the amount of granulation mixture encased within them.

Optional alternative step B: Granules can be packaged into four or eight ounce cups or bottles which are then sealed with a removable lid for eventual reconstitution with water, or other aqueous media, prior to administration. The dosage contained within each individual cup can be determined by the quantity (mass or volume) of granulation mixture placed in the cup prior to sealing. Optionally, the cup is purged of oxygen prior to sealing, and the seal is resistant to penetration by both oxygen and water vapor.

Optional alternative step C: Granules can be packaged in sealed tear-open sachets or packets made of laminated foil/plastic. The dosage to be administered can be determined by the quantity (mass or volume) of granulation mixture placed in each individual sachet/packet prior to sealing. Optionally, the sachet/packet is purged of oxygen prior to sealing, and is resistant to penetration by both oxygen and water vapor. To administer the dosage form, the sachet/packet is torn open and poured into a glass or cup. Water, or any other suitable liquid medium is then added to the glass or cup and the mixture is stirred to suspend and dissolve the granules. The entire resulting suspension is administered to the human subject in need of treatment. In this configuration, the suitable liquid media can be any suitable beverage, including plain water, and ideally is an acidic beverage such as a fruit juice or a carbonated drink such as a cola.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be understood that certain changes and modifications may be practiced within the scope of the present invention.

1. A method of treating diseases or disorders responsive to inhibition of Hsp90 in a human patient in need thereof, said method comprising orally administering to said human patient a therapeutically-effective amount of the compound (2S)-1-[4-(2-{6-amino-8-[(6-bromo-1,3-benzodioxol-5-yl)thio]-9H-purin-9-yl}ethyl)piperidin-1-yl]-1-oxopropan-2-ol, or a pharmaceutically-acceptable salt thereof.

2. The method of claim 1, wherein said treating diseases or disorders responsive to inhibition of Hsp90 comprises treating cancers.

3. (canceled)

4. The method of claim 2, wherein said cancers are selected from Hodgkin's disease, non-Hodgkin's lymphoma, acute lymphocytic leukemia, chronic lymphocytic leukemia, multiple myeloma, acute myelogenous leukemia, chronic myelogenous leukemia, myeloproliferative neoplasms, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, non-small cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, primary myelofibrosis, adrenal cortex carcinoma, skin cancer, prostatic carcinoma, and combinations thereof.

5. The method of claim 2, wherein said cancers comprise gastric cancer, colon cancer, prostate cancer, small-cell lung cancer, non-small cell lung cancer, ovarian cancer, acute myeloid leukemia, multiple myeloma, renal cell carcinoma, gastrointestinal stromal tumor, chronic myeloid leukemia, glioblastoma multiforme, astrocytomas, medulloblastomas, melanoma, breast cancer, pancreatic cancer, or combinations thereof.

6-7. (canceled)

8. The method of claim 2, said method further comprising administering to said human patient a therapeutically-effective amount of said compound, sufficient to provide in the human patient a plasma Cₘₐₓ ranging from about 1,500 ng/mL to about 30,000 ng/mL, or an amount of a pharmaceutically-acceptable salt of said compound sufficient to achieve an equimolar concentration in the plasma of the human patient.

9. The method of claim 8, wherein the Cₘₐₓ to be achieved with daily dosing ranges from about 6,000 ng/mL to about 30,000 ng/mL.

10. The method of claim 8, wherein the Cₘₐₓ to be achieved with twice daily dosing ranges from about 6,000 ng/mL to about 15,000 ng/mL.

11. The method of claim 2, said method further comprising administering to said human patient a therapeutically-effective amount of said compound, sufficient to provide in the human patient an AUC ranging from about 10,000 hr*ng/mL to about 700,000 hr*ng/mL, or an amount of a pharmaceutically-acceptable salt of said compound sufficient to achieve an equivalent exposure in the human patient.

12-14. (canceled)

15. The method of claim 11, wherein the AUC is calculated over a 24 hour interval, and wherein the AUC(0-24) to be achieved with a daily dose ranges from about 90,000 hr*ng/mL to about 400,000 hr*ng/mL.

16. The method of claim 11, wherein the AUC is calculated over an infinite time interval, and wherein the AUC(0-inf) to be achieved with a daily dose ranges from about 130,000 hr*ng/mL to about 700,000 hr*ng/mL.

17. The method of claim 11, wherein the AUC is calculated over a 12 hour interval, and wherein the AUC(0-12) to be achieved with a twice daily dose ranges from about 30,000 hr*ng/mL to about 80,000 hr*ng/mL.

18-19. (canceled)

20. The method claim 2, wherein the therapeutically-effective amount ranges from about 50 mg/m² to about 600 mg/m², per day.

21-23. (canceled)

24. The method of claim 2, wherein the therapeutically-effective amount ranges from about 50 mg/m² to about 600 mg/m², twice-a-day.

25-27. (canceled)

28. The method of claim 2, wherein the therapeutically-effective amount ranges from about 100 mg to about 1000 mg, per day.

29-31. (canceled)

32. The method of claim 2, wherein the therapeutically-effective amount ranges from about 25 mg to about 1000 mg, twice-per-day.

33-35. (canceled)

36. The method of claim 2, wherein administration results in at least about a 50% regression in tumor volume.

37. The method of claim 2, wherein administration results in at least about a 50% inhibition of tumor growth.

38-40. (canceled)

41. A method of treating or preventing diseases or disorders responsive to inhibition of Hsp90 in a human patient in need thereof, said method comprising administering to said human patient a therapeutically-effective amount of the compound (2S)-1-[4-(2-{6-amino-8-[(6-bromo-1,3-benzodioxol-5-yl)thio]-9H-purin-9-yl}ethyl)piperidin-1-yl]-1-oxopropan-2-ol, sufficient to provide in the human patient a plasma Cₘₐₓ ranging from about 1,500 ng/mL to about 30,000 ng/mL, or an amount of a pharmaceutically-acceptable salt of said compound, sufficient to achieve an equimolar concentration in the plasma of the human patient.

42. The method of claim 41, wherein said compound is administered orally.

43. A method of treating or preventing diseases or disorders responsive to inhibition of Hsp90 in a human patient in need thereof, said method comprising administering to said human patient a therapeutically-effective amount of the compound (2S)-1-[4-(2-{6-amino-8-[(6-bromo-1,3-benzodioxol-5-yl)thio]-9H-purin-9-yl}ethyl)piperidin-1-yl]-1-oxopropan-2-ol, sufficient to provide in the human patient an AUC ranging from about 10,000 hr*ng/mL to about 700,000 hr*ng/mL, or an amount of a pharmaceutically-acceptable salt of said compound, sufficient to achieve an equivalent exposure in the human patient.

44. The method of claim 43, wherein said compound is administered orally.

45. A pharmaceutical composition comprising the compound (2S)-1-[4-(2-{6-amino-8-[(6-bromo-1,3-benzodioxol-5-yl)thio]-9H-purin-9-yl}ethyl)piperidin-1-yl]-1-oxopropan-2-ol, or a pharmaceutically-acceptable salt thereof, and at least one pharmaceutically-acceptable solubilizing agent.

46. The pharmaceutical composition of claim 45, wherein said at least one pharmaceutically-acceptable solubilizing agent comprises a pharmaceutically-acceptable cyclodextrin.

47. The pharmaceutical composition of claim 45, wherein the pharmaceutically-acceptable cyclodextrin comprises a beta-cyclodextrin.

48. The pharmaceutical composition of claim 47, wherein said beta-cyclodextrin comprises a hydroxypropyl beta-cyclodextrin (HPbCD) or a sulfobutylether beta-cyclodextrin (SBEbCD).

49-58. (canceled)

59. A solid pharmaceutical dosage form comprising the pharmaceutical composition of claim 48 and at least one solid pharmaceutically-acceptable excipient.

60. The solid pharmaceutical dosage form of claim 59, wherein said at least one solid pharmaceutically-acceptable excipient comprises at least one binder, at least diluent, at least one tableting agent, at least one flavoring agent, at least one sweetening agent, or at least one coating agent, or combinations thereof.

61. (canceled)

62. A method of making a pharmaceutical dosage form, said method comprising:
mixing the compound (2S)-1-[4-(2-{6-amino-8-[(6-bromo-1,3-benzodioxol-5-yl)thio]-9H-purin-9-yl}ethyl)piperidin-1-yl]-1-oxopropan-2-ol, or a pharmaceutically-acceptable salt thereof, with a pharmaceutically-acceptable cyclodextrin and dissolving the mixture in an aqueous solvent to form a solution.

63-64. (canceled)

65. The method of claim 62, further comprising granulating said solution with at least one binder and at least one diluent to form granules.

66. The method of claim 65, wherein said granulating comprises using a fluid bed process.

67-90. (canceled)
